(19) United States
(12) Patent Application Publication
(10) Pub. No.: US 2014/0148412 A1
(43) Pub. Date: May 29, 2014

(54) NOVEL 17B-HETEROARYL-SUBSTITUTED STEROIDS AS MODULATORS OF GABAA RECEPTORS

(71) Applicant: THE REGENTS OF THE UNIVERSITY OF CLAIFORNIA, Oakland, CA (US)

(72) Inventor: Derk J. Hogenkamp, Carlsbad, CA (US)

(73) Assignee: THE REGENTS OF THE UNIVERSITY OF CLAIFORNIA, Oakland, CA (US)

(21) Appl. No.: 14/166,912

(22) Filed: Jan. 29, 2014

Related U.S. Application Data
(63) Continuation of application No. PCT/US2012/048816, filed on Jul. 30, 2012.
(60) Provisional application No. 61/513,059, filed on Jul. 29, 2011.

(30) Foreign Application Priority Data
Jul. 30, 2012 (US) PCT/US2012/048816

(51) Int. Cl. C07J 43/00

(52) U.S. Cl. CPC C07J 43/003
USPC 514/63; 548/247; 514/176; 548/248; 548/110; 546/121; 548/193

(57) ABSTRACT

The invention is directed to novel 17β-heteroaryl substituted steroids of Formula I, pharmaceutical compositions thereof, and their use as modulators of GABA_{A} receptors.

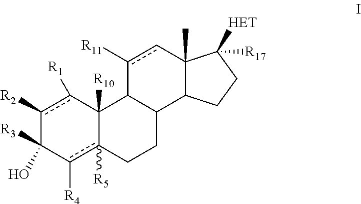

### RELATED APPLICATIONS

This application is a continuation of International Application No. PCT/US2012/048816, filed on Jul. 30, 2012, which claims priority to U.S. provisional application Ser. No. 61/513,059, filed on Jul. 29, 2011, each of which is incorporated by reference in its entirety.

### FIELD OF THE INVENTION

Novel 17β-heteroaryl-substituted steroid compounds are described together with methods of using the compounds for treating CNS conditions. Their pharmaceutical compositions are also described.

### BACKGROUND OF THE INVENTION

GABA is the major inhibitory neurotransmitter in the mammalian CNS. The binding of GABA to its site on the GABA_{A} receptor (GABA_{A}R) is influenced by allosteric modulators that include benzodiazepines (BZs), barbiturates and neuroactive steroids. GABA_{A} receptors are members of the Cys-loop family that include the GABAc, α7 subtype of nicotinic acetylcholine receptors (nAChR), glycine and 5-hydroxytryptamine type-3 (5-HT₃) receptors. The receptor is a heteropentamer that is generally composed of two α, two β and one γ or δ-subunit that form an ionophore that passes chloride ions. The adult brain contains predominately the α₁β₁γ₂ subunit combination (60%) with the majority of the remaining receptors expressing α₂β₃γ₂ and α₃βₙγ₂ subunits (35%). Modulators of GABA_{A} receptors have found use as anxiolytics, anticonvulsants, anesthetics and as sedative-hypnotics. More recently, modulators were found to be useful in treating pain, depression and schizophrenia. Endogenous neuroactive steroids that bind to the GABA_{A}R are metabolites of progesterone and deoxycorticosterone that possess 3α-hydroxyl and 20-ketone groups. The metabolites include 3α-hydroxy-5α- and 5β-pregnan-20-one (3α,5α- and 5β-P) as well as 3α,21-dihydroxy-5α- and 5β-pregnan-20-one (5α- and 5β-THDOC). 3α,20α- and 20β-dihydroxypregnanes are also formed in the body. The corresponding 3β-hydroxyl epimers are inactive as modulators, indicating that the interaction of the neuroactive steroids with the receptor is a specific one, and not simply the result of changes in membrane fluidity. Synthetic analogs of these compounds have been developed that maintain activity for the receptor, but unlike the naturally occurring compounds, are orally active. Because they generally are 20-ketosteroids, the synthetic compounds often have poor pharmacokinetic (PK) characteristics, including short half-lives. The novel neuroactive steroids of the current invention lack the 20-ketone of the naturally occurring steroids and may have improved PK profiles as a result.

### SUMMARY OF THE INVENTION

The current invention is related to the observation that 17β-heteroaryl-substituted steroids of Formula I are modulators of GABA_{A} receptors and act to enhance GABA-facilitated chloride flux mediated through the GABA_{A} receptor complex (GRC).

The invention is related to treating disorders responsive to enhancement of GABA action on GABA_{A} receptors in a mammal by administrating an effective amount of a compound of Formula I as described herein.

The compounds of the present invention, being ligands for GABA_{A} receptors, are therefore of use in the treatment and/or prevention of a variety of disorders of the CNS. In one aspect, the compounds of the invention are useful in the treatment and/or prevention of disorders of the CNS involving neuronal hyperexcitiability. Such disorders include but are not limited to anxiety disorders, such as panic disorder, with or without agoraphobia, agoraphobia without history of panic disorder, animal and other phobias, including social phobias, obsessive-compulsive disorder, stress disorders including post-traumatic and acute stress disorder, and generalized or substance-induced anxiety disorder, neuroses, convulsions, epilepsy and other seizure disorders, migraine, and depressive or bipolar disorders, for example single episode or recurrent major depressive disorder, dysthymic disorder, bipolar I and bipolar II manic disorders, and cyclothymic disorder. Such disorders also include insomnia and other sleep disorders, including those involving reduced wakefulness such as narcolepsy and idiopathic hypersomnia. Other uses include treating cognitive dysfunction associated with schizophrenia and senile demenitas. Compounds of the invention are also useful as anesthetics, and can be used to treat chronic and acute pain. The compounds of the present invention are also of use in the treatment of depression and other affective disorders, and autism spectrum disorders (e.g., Fragile X syndrome, Aspergers syndrome, Rett syndrome etc.). Compounds of the invention are also useful for neurogenesis/neuroprotection after traumatic brain injury, in neuroinflammatory, neurodegenerative diseases (e.g., multiple sclerosis) and in Alzheimer's disease and in stroke.

Another aspect of the current invention is to provide a pharmaceutical composition useful for treating disorders responsive to the enhancement of GABA-facilitated chloride flux mediated through the GRC, containing an effective amount of a compound of Formula I, pharmaceutically acceptable salts, solvates, or prodrugs thereof, in a mixture of one or more pharmaceutically acceptable carriers or diluents.

Compounds useful in the present invention have not been heretofore reported. Thus, the present invention is also directed to novel substituted steroids having the structure of Formula I and pharmaceutically acceptable salts, solvates, or prodrugs thereof.

Further, the present invention is directed to ²H, ³H, ¹⁴C, ¹⁸F, ³⁵S, ³⁶Cl and ¹²⁵I isotopically labeled compounds of Formula I and their use as stable isotope analogs or their use as radioligands for their binding site on the GRC.

The present invention includes isomers of compounds of Formula I. Examples of such isomers include, for example, E and Z isomers of double bonds, enantiomers, and diastereomers.

Additional embodiments and advantages will be set forth in part in the description that follows, and in part will be obvious from the description, or may be learned by practice of the invention. The embodiments and advantages will be realized and attained by means of the elements and combinations particularly pointed out in the appended claims.

It is to be understood that both the foregoing general description and the following detailed description are exemplary and explanatory only and are not restrictive of the invention, as claimed.

All publications and patent applications mentioned in this specification are incorporated by reference in their entirety to the same extent as if each individual publication or patent application was specifically and individually indicated to be incorporated by reference.

### DETAILED DESCRIPTION OF THE INVENTION

In one embodiment, there are provided substituted steroids represented by Formula I:

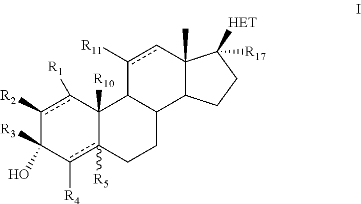

or a pharmaceutically acceptable salt, solvate, or prodrug thereof, wherein:

each R₁, R₂, R₃, R₄, and R₁₇ is independently selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁;

R₅ is a 5α or 5β-hydrogen, fluorine or absent if there is a C4-C5 double bond; R₁₀ is hydrogen, fluorine or methyl;

R₁₁ is hydrogen, a hydroxyl, an NR₂₃R₂₄ group or a keto group;

each R₁₉ independently is hydrogen, halogen, C₁₋₈ alkyl, C₂₋₈ alkenyl, C₂₋₈ alkynyl, C₁₋₄ acyl, —C(═O)OC₁₋₄ alkyl, —C(═O)H, —Si(C₁₋₄ alkyl)₃, or C₁₋₈ haloalkyl, wherein each of said alkyl, alkenyl, alkynyl, and haloalkyl is optionally substituted with 1-5 R₂₁;

R₂₀ is selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁; or

R₂₀ is selected from the group consisting of aryl, heteroaryl, C₃₋₆ cycloalkyl, C₄₋₆ cycloalkenyl, heterocycloalkyl or heterocycloalkenyl, wherein said heterocycloalkyl is optionally fused with phenyl or a 5-6 membered heteroaryl having 1-3 heteroatoms, wherein one or more of the carbon atoms in said heterocycloalkyl or heterocycloalkenyl optionally may be oxidized to C(═O), wherein each of said aryl, heteroaryl, cycloalkyl, cycloalkenyl, heterocycloalkyl, and heterocycloalkenyl is optionally substituted with 1-5 R₂₂; or

R₁₉ and R₂₀ taken together with the atoms to which they are attached form a heteroaryl, a heterocycloalkyl or a heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), wherein each of said heteroaryl, heterocycloalkyl, and heterocycloalkenyl is optionally substituted with 1-5 R₂₂;

each R₂₁ is independently selected from the group consisting of hydroxyl, C₁₋₆ alkoxy, C₁₋₈ haloalkoxy, C₃₋₆ cycloalkoxy, NR₂₃R₂₄, aryl, heteroaryl, C₃₋₆ cycloalkyl, C₄₋₆ cycloalkenyl, heterocycloalkyl or heterocycloalkenyl, wherein each of said aryl, heteroaryl, cycloalkyl and cycloalkenyl are optionally substituted with 1-5 R₂₂; and wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), and wherein each of said heterocycloalkyl and said heterocycloalkenyl is optionally substituted with 1-5 R₂₂;

each R₂₂ is independently selected from the group consisting of nitro, nitrile, hydroxyl, halogen, C₁₋₆ acyl, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₆ cycloalkoxy, aryl, heteroaryl, —NR₂₃R₂₄, —C(═O)OR₂₃, —C(═O)NHR₂₃, —NHC(═O)R₂₅, —NHS(═O)₂R₂₅, —S(═O)₀₋₂R₂₅, —S(═O)₂NHR₂₃, C₃₋₆ cycloalkyl, C₄₋₆ cycloalkenyl, heterocycloalkyl or heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O);

each of R₂₃ and R₂₄ is independently hydrogen, C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted with 1-5 R₂₁;

R₂₅ is C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted;

HET is a heteroaryl group selected from

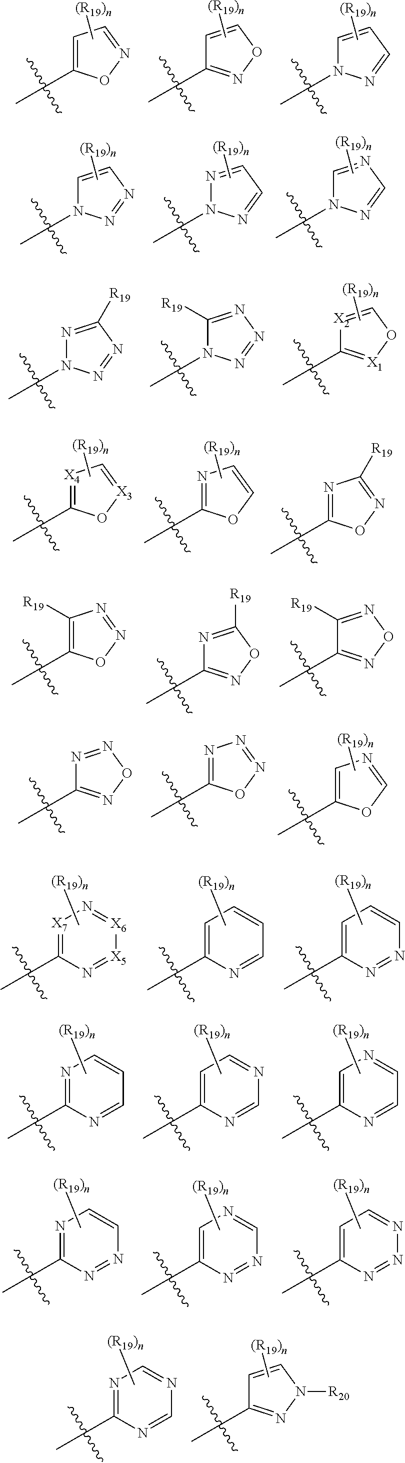

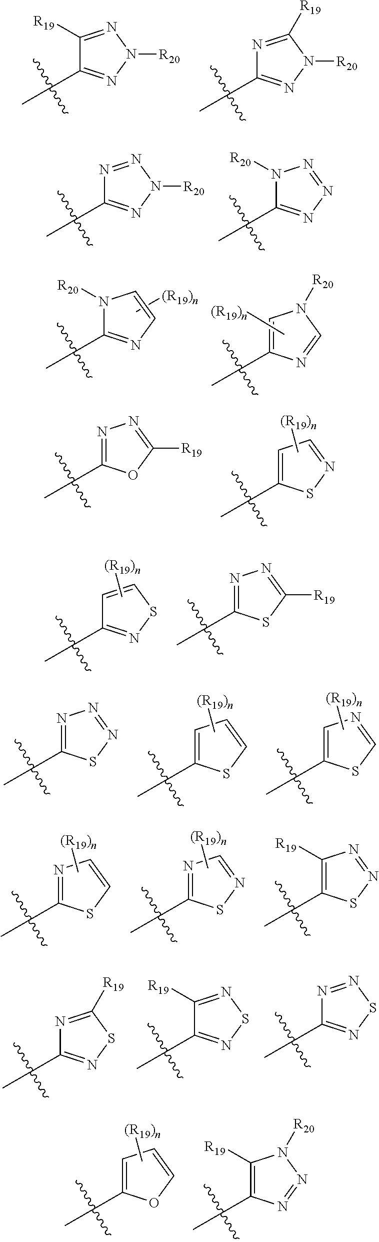

when X₁ is N, X₂ is CR₁₉ or N, when X₁ is CR₁₉, X₂ is N;

X₃ and X₄ are independently CR₁₉ and N;

when X₅ is N, X₆ and X₇ are independently CR₁₉; or

when X₆ is N, X₅ and X₇ are independently CR₁₉; or

when X₇ is N, X₅ and X₆ are independently CR₁₉;

n is an integer from 1 to 4;

the dashed lines represent optional double bonds; with the proviso that when the bond between C1 and C2 is a single bond, then R₂ and R₃ are not both hydrogen.

Compounds useful in another aspect of the invention include compounds of Formula II:

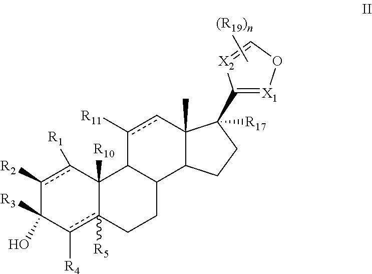

where R₁, R₂, R₃, R₄, R₅, R₁₀, R₁₁, R₁₇, R₁₉, R₂₁, R₂₂, R₂₃, R₂₄, and R₂₅ are defined as above and when X₁ is N, X₂ is CR₁₉ or N, when X₁ is CR₁₉, X₂ is N, n is an integer from 1 to 2, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula III:

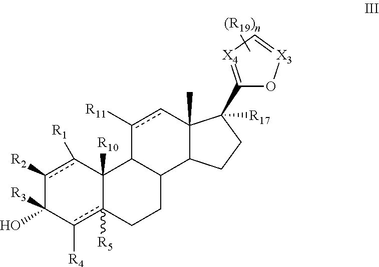

where R₁, R₂, R₃, R₄, R₅, R₁₀, R₁₁, R₁₇, R₁₉, R₂₁, R₂₂, R₂₃, R₂₄, and R₂₅ are defined as above and X₃ and X₄ are independently CR₁₉ or N, n is an integer from 1 to 3, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula IV:

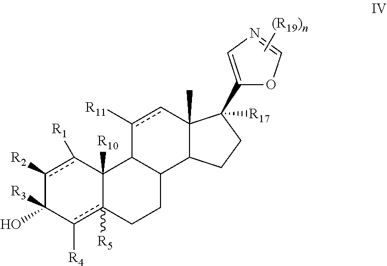

where R₁, R₂, R₃, R₄, R₅, R₁₀, R₁₁, R₁₇, R₁₉, R₂₁, R₂₂, R₂₃, R₂₄, and R₂₅ are defined as above, n is 1 or 2, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula V:

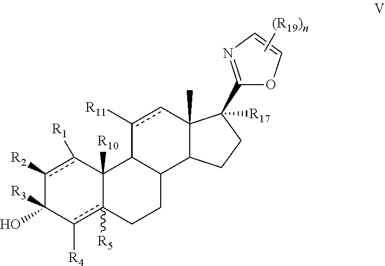

where R₁, R₂, R₃, R₄, R₅, R₁₀, R₁₁, R₁₇, R₁₉, R₂₁, R₂₂, R₂₃, R₂₄, and R₂₅ are defined as above, n is 1 or 2, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula VI:

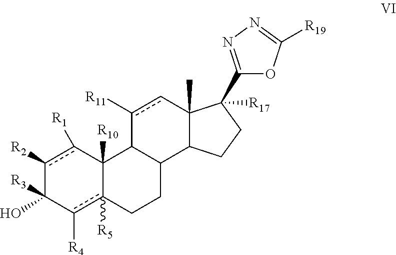

where R₁, R₂, R₃, R₄, R₅, R₁₀, R₁₁, R₁₇, R₁₉, R₂₁, R₂₂, R₂₃, R₂₄, and R₂₅ are defined as above the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula VII:

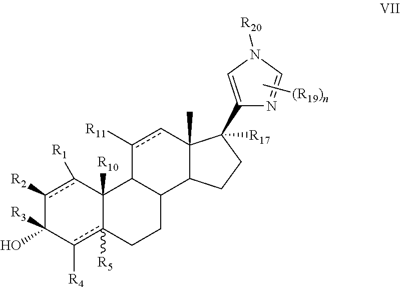

where R₁, R₂, R₃, R₄, R₅, R₁₀, R₁₁, R₁₇, R₁₉, R₂₀, R₂₁, R₂₂, R₂₃, R₂₄, and R₂₅ are defined as above, n is 1 or 2, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula VIII:

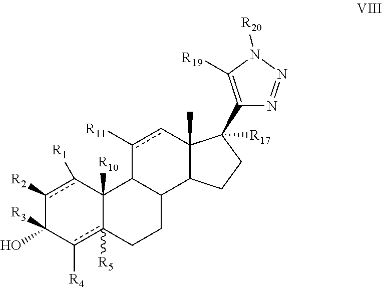

where R₁, R₂, R₃, R₄, R₅, R₁₀, R₁₁, R₁₇, R₁₉, R₂₀, R₂₁, R₂₂, R₂₃, R₂₄, and R₂₅ are defined as above, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula IX:

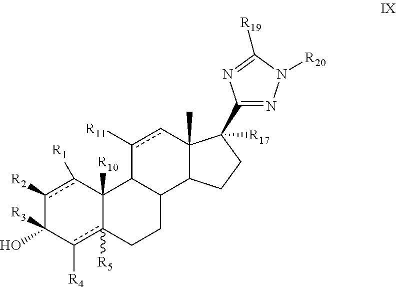

where R₁, R₂, R₃, R₄, R₅, R₁₀, R₁₁, R₁₇, R₁₉, R₂₀, R₂₁, R₂₂, R₂₃, R₂₄, and R₂₅ are defined as above, the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula X:

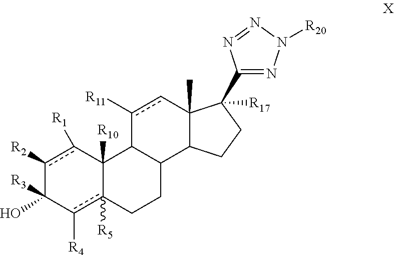

where R₁, R₂, R₃, R₄, R₅, R₁₀, R₁₁, R₁₇, R₂₀, R₂₁, R₂₂, R₂₃, R₂₄, and R₂₅ are defined as above the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula XI:

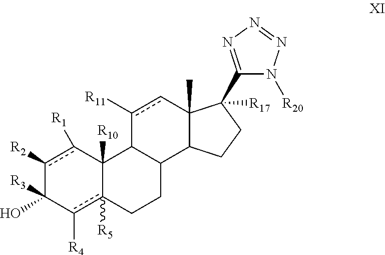

where R₁, R₂, R₃, R₄, R₅, R₁₀, R₁₁, R₁₇, R₂₀, R₂₁, R₂₂, R₂₃, R₂₄, and R₂₅ are defined as above the dashed lines represent optional double bonds; with the proviso that when the bond between C1 and C2 is a single bond, then R₂ and R₃ are not both hydrogen, and pharmaceutically acceptable salts, solvates, or prodrugs thereof.

Compounds useful in another aspect of the invention include compounds of Formula XII and pharmaceutically acceptable salts, solvates, or prodrugs thereof:

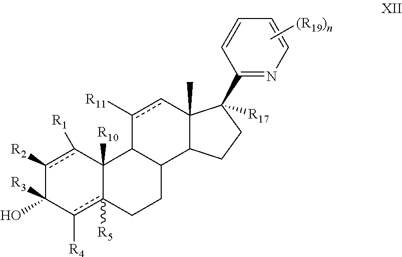

where R₁, R₂, R₃, R₄, R₅, R₁₀, R₁₁, R₁₇, R₁₉, R₂₁, R₂₂, R₂₃, R₂₄, and R₂₅ are defined as above, n is an integer from 1 to 4, the dashed lines represent optional double bonds; with the proviso that when the bond between C1 and C2 is a single bond, then R₂ and R₃ are not both hydrogen.

Compounds useful in another aspect of the invention include compounds of Formula XIII and pharmaceutically acceptable salts, solvates, or prodrugs thereof:

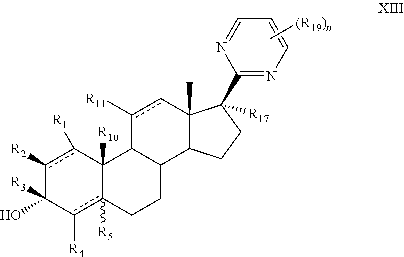

where R₁, R₂, R₃, R₄, R₅, R₁₀, R₁₁, R₁₇, R₁₉, R₂₁, R₂₂, R₂₃, R₂₄, and R₂₅ are defined as above, and n is an integer from 1 to 3, the dashed lines represent optional double bonds; with the proviso that when the bond between C1 and C2 is a single bond, then R₂ and R₃ are not both hydrogen.

Compounds useful in another aspect of the invention include compounds of Formula XIV and pharmaceutically acceptable salts, solvates, or prodrugs thereof:

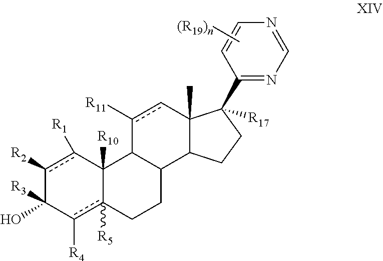

where R₁, R₂, R₃, R₄, R₅, R₁₀, R₁₁, R₁₇, R₁₉, R₂₁, R₂₂, R₂₃, R₂₄, and R₂₅ are defined as above, and n is an integer from 1 to 3, the dashed lines represent optional double bonds; with the proviso that when the bond between C1 and C2 is a single bond, then R₂ and R₃ are not both hydrogen.

Compounds useful in another aspect of the invention include compounds of Formula XV and pharmaceutically acceptable salts, solvates, or prodrugs thereof:

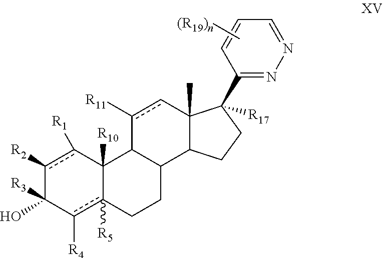

where R₁, R₂, R₃, R₄, R₅, R₁₀, R₁₁, R₁₇, R₁₉, R₂₁, R₂₂, R₂₃, R₂₄, and R₂₅ are defined as above, and n is an integer from 1 to 3, the dashed lines represent optional double bonds; with the proviso that when the bond between C1 and C2 is a single bond, then R₂ and R₃ are not both hydrogen.

Compounds useful in another aspect of the invention include compounds of Formula XVI and pharmaceutically acceptable salts, solvates, or prodrugs thereof:

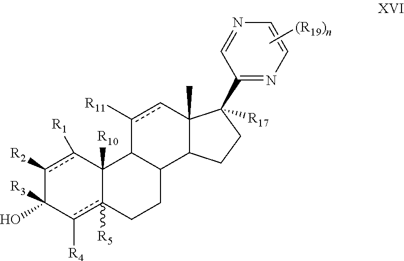

where R₁, R₂, R₃, R₄, R₅, R₁₀, R₁₁, R₁₇, R₁₉, R₂₁, R₂₂, R₂₃, R₂₄, and R₂₅ are defined as above, and n is an integer from 1 to 3, the dashed lines represent optional double bonds; with the proviso that when the bond between C1 and C2 is a single bond, then R₂ and R₃ are not both hydrogen.

Compounds useful in another aspect of the invention include compounds of Formula XVII and pharmaceutically acceptable salts, solvates, or prodrugs thereof:

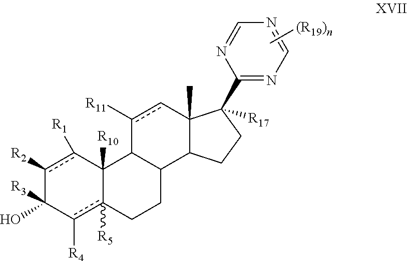

where R₁, R₂, R₃, R₄, R₅, R₁₀, R₁₁, R₁₇, R₁₉, R₂₁, R₂₂, R₂₃, R₂₄, and R₂₅ are defined as above, and n is 1 or 2, the dashed lines represent optional double bonds; with the proviso that when the bond between C1 and C2 is a single bond, then R₂ and R₃ are not both hydrogen.

Compounds useful in another aspect of the invention include compounds of Formula XVIII:

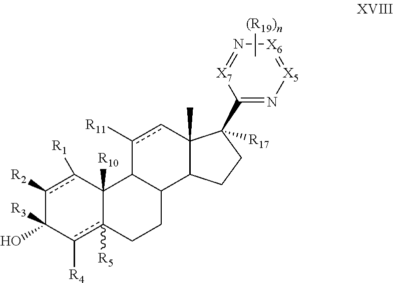

where R₁, R₂, R₃, R₄, R₅, R₁₀, R₁₁, R₁₇, R₁₉, R₂₁, R₂₂, R₂₃, R₂₄, and R₂₅ are defined as above;

n is an integer from 1 to 2;

X₅ is N, X₆ and X₇ are independently CR₁₉; or

X₆ is N, X₅ and X₇ are independently CR₁₉; or

X₇ is N, X₅ and X₆ are independently CR₁₉,

the dashed lines represent optional double bonds; with the proviso that when the bond between C1 and C2 is a single bond, then R₂ and R₃ are not both hydrogen and pharmaceutically acceptable salts, solvates, or prodrugs thereof.

In each of the above Formulae I-XVIII, R₁, R₂, R₄, R₅, R₁₇, R₂₀, R₂₂, R₂₃, R₂₄, and R₂₅ are hydrogen, R₃ is selected from the group of C1-4 alkyl, and C1-4 haloalkyl; R₁₉ is independently hydrogen, halogen, optionally substituted C1-4 alkyl, and C1-4 haloalkyl; C1 to C2, C4 to C5, and C11 to C12 are single bonds, and pharmaceutically acceptable salts, solvates, or prodrugs thereof.

Compounds useful in another aspect of the invention include compounds of each of Formulae I-XVIII, wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, and C1-4 alkyl, optionally substituted with hydroxy, and halogen; C1 to C2, C4 to C5, and C11 to C12 are single bonds, and pharmaceutically acceptable salts, solvates, or prodrugs thereof. In another aspect of such compounds, R₃ is methyl, or trifluoromethyl; R₁₀ is methyl; R₁₉ is independently hydrogen, C1-4 alkyl and hydroxymethyl, and pharmaceutically acceptable salts, solvates, or prodrugs thereof.

In another embodiment of the invention, the compounds include compounds of each of Formulae I-III wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, halogen, C1-4 alkyl, optionally substituted with hydroxy, and halogen; HET is selected from the group consisting of 5-isoxazolyl, 3-isoxazolyl, 2-oxazolyl, 4-oxazolyl and 5-oxazolyl, all optionally substituted with 1 to 2 R₁₉ groups; C1 to C2, C4 to C5, and C11 to C12 are single bonds, and pharmaceutically acceptable salts, solvates, or prodrugs thereof. In another aspect of such compounds, R₃ is methyl; R₅ is a 5α-hydrogen atom; R₁₀ is methyl; R₁₉ is independently hydrogen, C1-4 alkyl and hydroxymethyl; and pharmaceutically acceptable salts, solvates, or prodrugs thereof.

In another embodiment of the invention, the compounds includes compounds of each of Formulae I-III wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, halogen, C1-4 alkyl, optionally substituted with hydroxy, and halogen; HET is selected from the group consisting of 5-isoxazolyl and 3-isoxazolyl, all optionally substituted with 1 to 2 R₁₉ groups; C1 to C2, C4 to C5, and C11 to C12 are single bonds, and pharmaceutically acceptable salts, solvates, or prodrugs thereof. In another aspect of such compounds, R₃ is trifluoromethyl; R₅ is a 5β-hydrogen atom; R₁₀ is hydrogen or methyl; R₁₉ is independently hydrogen, C1-4 alkyl and hydroxymethyl; and pharmaceutically acceptable salts, solvates, or prodrugs thereof.

Preferred compounds of the invention are selected from the following:
5-[3α-Hydroxy-3β-methyl-5α-androstan-17β-yl]-isoxazole;
3-[3α-hydroxy-3β-methyl-5α-androstan-17β-yl]-isoxazole;
ethyl 5-[3α-hydroxy-3β-methyl-5α-androstan-17β-yl]-3-isoxazolecarboxylate;
5-[3α-hydroxy-3β-methyl-5α-androstan-17β-yl]-3-(hydroxymethyl)isoxazole;
5-[3α-hydroxy-3β-methyl-5α-androstan-17β-yl]-3-isoxazolecarboxaldehyde;
(S)-3-[3α-hydroxy-3β-methyl-5α-androstan-17β-yl]-5-(1-hydroxyethyl)isoxazole;
3-[3α-hydroxy-3β-methyl-5α-androstan-17β-yl]-5-(hydroxymethyl)isoxazole;
3-[3α-hydroxy-3β-methyl-5α-androstan-17β-yl]-5-(2-hydroxy-2-propyl)isoxazole;
3-[3α-hydroxy-3β-methyl-5α-androstan-17β-yl]-5-(trimethylsilyl)isoxazole; and
2-[3α-hydroxy-3β-methyl-5α-androstan-17β-yl]-imidazo[1,2-a]pyridine and pharmaceutically acceptable salts, solvates, or prodrugs thereof.

Another aspect of the invention is pharmaceutical compositions of the various compounds of Formulae I-XVIII as described above in various aspects and embodiments with a pharmaceutically acceptable excipient.

Another aspect of the invention is pharmaceutical compositions of the various compounds of Formulae I-XVIII, R₁, R₂, R₄, R₅, R₁₇, R₂₀, R₂₂, R₂₃, R₂₄, and R₂₅ are hydrogen, R₃ is selected from the group of C1-4 alkyl, and C1-4 haloalkyl; R₁₉ is independently hydrogen, halogen, optionally substituted C1-4 alkyl, and C1-4 haloalkyl; C1 to C2, C4 to C5, and C11 to C12 are single bonds, and pharmaceutically acceptable salts, solvates, or prodrugs thereof, with a pharmaceutically acceptable excipient.

Pharmaceutical composition useful in another aspect of the invention include compounds of each of Formulae I-XVIII, wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, and C1-4 alkyl, optionally substituted with hydroxy, and halogen; C1 to C2, C4 to C5, and C11 to C12 are single bonds, and pharmaceutically acceptable salts, solvates, or prodrugs thereof with a pharmaceutically acceptable excipient. In another aspect of such compounds, R₃ is methyl, or trifluoromethyl; R₁₀ is methyl; R₁₉ is independently hydrogen, C1-4 alkyl and hydroxymethyl, and pharmaceutically acceptable salts, solvates, or prodrugs thereof with a pharmaceutically acceptable excipient.

In another embodiment of the invention, the pharmaceutical compositions include compounds of each of Formulae I-III wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, halogen, C1-4 alkyl, optionally substituted with hydroxy, and halogen; HET is selected from the group consisting of 5-isoxazolyl, 3-isoxazolyl, 2-oxazolyl, 4-oxazolyl and 5-oxazolyl, all optionally substituted with 1 to 2 R₁₉ groups; C1 to C2, C4 to C5, and C11 to C12 are single bonds, and pharmaceutically acceptable salts, solvates, or prodrugs thereof with a pharmaceutically acceptable excipient. In another aspect of such pharmaceutical compositions, R₃ is methyl; R₅ is a 5α-hydrogen atom; R₁₀ is methyl; R₁₉ is independently hydrogen, C1-4 alkyl and hydroxymethyl; and pharmaceutically acceptable salts, solvates, or prodrugs thereof with a pharmaceutically acceptable excipient.

In another embodiment of the invention, the pharmaceutical compositions includes compounds of each of Formulae I-III wherein R₁, R₂, R₄, R₅, R₁₁, R₁₇, R₂₂, R₂₃, R₂₄, and R₂₅ are hydrogen; R₃ is selected from the group consisting of C1-4 alkyl, C2-4 alkenyl, C2-4 alkynyl, and C1-4 haloalkyl; R₁₀ is hydrogen or methyl; R₁₉ is independently hydrogen, halogen, C1-4 alkyl, optionally substituted with hydroxy, and halogen; HET is selected from the group consisting of 5-isoxazolyl and 3-isoxazolyl, all optionally substituted with 1 to 2 R₁₉ groups; C1 to C2, C4 to C5, and C11 to C12 are single bonds, and pharmaceutically acceptable salts, solvates, or prodrugs thereof and a pharmaceutically acceptable excipient. In another aspect of such pharmaceutical compositions, R₃ is trifluoromethyl; R₅ is a 5β-hydrogen atom; R₁₀ is hydrogen or methyl; R₁₉ is independently hydrogen, C1-4 alkyl and hydroxymethyl; and pharmaceutically acceptable salts, solvates, or prodrugs thereof and a pharmaceutically acceptable excipient.

Preferred pharmaceutical compositions include compounds of the invention selected from the following:
5-[3α-Hydroxy-3β-methyl-5α-androstan-17β-yl]-isoxazole;
3-[3α-hydroxy-3β-methyl-5α-androstan-17β-yl]-isoxazole;
ethyl 5-[3α-hydroxy-3β-methyl-5α-androstan-17β-yl]-3-isoxazolecarboxylate;
5-[3α-hydroxy-3β-methyl-5α-androstan-17β-yl]-3-(hydroxymethyl)isoxazole;
5-[3α-hydroxy-3β-methyl-5α-androstan-17β-yl]-3-isoxazolecarboxaldehyde;
(S)-3-[3α-hydroxy-3β-methyl-5α-androstan-17β-yl]-5-(1-hydroxyethyl)isoxazole;
3-[3α-hydroxy-3β-methyl-5α-androstan-17β-yl]-5-(hydroxymethyl)isoxazole;
3-[3α-hydroxy-3β-methyl-5α-androstan-17β-yl]-5-(2-hydroxy-2-propyl)isoxazole;
3-[3α-hydroxy-3β-methyl-5α-androstan-17β-yl]-5-(trimethylsilyl)isoxazole; and
2-[3α-hydroxy-3β-methyl-5α-androstan-17β-yl]-imidazo[1,2-a]pyridine and pharmaceutically acceptable salts, solvates, or prodrugs thereof with a pharmaceutically acceptable excipient.

Another aspect of the invention is the use of the various compounds of Formulae I-XVIII as described above in various aspects and embodiments including such compounds where the bond between C1 and C2 is a single bond and R₂ and R₃ are both hydrogen, to treat or prevent disorders of the CNS involving neuronal hyperexcitiability. Such disorders include but are not limited to anxiety disorders, such as panic disorder, with or without agoraphobia, agoraphobia without history of panic disorder, animal and other phobias, including social phobias, obsessive-compulsive disorder, stress disorders including post-traumatic and acute stress disorder, and generalized or substance-induced anxiety disorder, neuroses, convulsions, epilepsy and other seizure disorders, migraine, and depressive or bipolar disorders, for example single episode or recurrent major depressive disorder, dysthymic disorder, bipolar I and bipolar II manic disorders, and cyclothymic disorder. Such disorders also include insomnia and other sleep disorders, including those involving reduced wakefulness such as narcolepsy and idiopathic hypersomnia. Other uses include treating cognitive dysfunction associated with schizophrenia and senile demenitas. Compounds of the invention are also useful as anesthetics, and can be used to treat chronic and acute pain. The compounds of the present invention are also of use in the treatment of depression and other affective disorders, and autism spectrum disorders (e.g., Fragile X syndrome, Aspergers syndrome, Rett syndrome etc.). In another embodiment the compounds are used to treat anxiety, epilepsy and other seizure disorders, insomnia, depression, cognitive dysfunction in schizophrenia, cognitive impairment after traumatic brain injury, and for neurogenesis/neuroprotection after traumatic brain injury, in neuroinflammatory, neurodegenerative diseases (e.g., multiple sclerosis) and in Alzheimer's disease and in stroke.

In one embodiment, the compounds of the invention are used to treat epilepsy. In one embodiment, the compounds of the invention are used to treat anxiety. In one embodiment, the compounds of the invention are used to treat depression. In one embodiment, the compounds of the invention are used to treat pain. In one embodiment, the compounds of the invention are used to treat insomnia. In one embodiment, the compounds of the invention are used to treat schizophrenia.

### DEFINITIONS

Unless specifically noted otherwise herein, the definitions of the terms used are standard definitions used in the art of organic synthesis and pharmaceutical sciences.

The term “administering” as used herein can include providing to one or more subjects by any means. Unless otherwise limited, the one or more subjects can be members of any one or more species, such as human or non-human animals. Unless otherwise limited, administering can be accomplished by any route, including without limitation the routes discussed below. Administering can also include perscribing or filling a perscription for a compound or formulation.

The term “halogen” as used herein refers to a halogen radical selected from fluoro, chloro, bromo and iodo.

The term “keto” refers ═O.

The term “nitrile” refers to —C≡N.

The term “nitro” refers to —NO₂.

The term “alkyl” refers to a saturated aliphatic hydrocarbon radical. “Alkyl” refers to both branched and unbranched alkyl groups. Examples of “alkyl” include alkyl groups that are straight chain alkyl groups containing from one to eight carbon atoms and branched alkyl groups containing from three to eight carbon atoms. “Alkyl” includes but is not limited to straight chain alkyl groups containing from one to six carbon atoms and branched alkyl groups containing from three to six carbon atoms. This term is exemplified by groups such as methyl, ethyl, n-propyl, 1-methylethyl (isopropyl), 1,1-dimethylethyl (tert-butyl), and the like. It may be abbreviated “Alk”. It should be understood that any combination term using an “alk” or “alkyl” prefix refers to analogs according to the above definition of “alkyl” including the number of carbon atoms. For example, terms such as “alkoxy”, “alkylthio”, “alkylamino” refer to alkyl groups linked to a second group via an oxygen, sulfur, or nitrogen atom, respectively.

The term “haloalkyl” refers to an alkyl group in which one or more hydrogen atoms are replaced with halogen atoms. This term includes but is not limited to perhaloalkyl groups such as trifluoromethyl. In one embodiment the haloalkyl groups are alkyl groups substituted with one or more fluoro or chloro. The term “haloalkoxy” refers to haloalkyl groups linked to a second group via an oxygen atom.

The term “alkenyl” refers to a mono or polyunsaturated aliphatic hydrocarbon radical. The mono or polyunsaturated aliphatic hydrocarbon radical contains at least one carbon-carbon double bond. “Alkenyl” refers to both branched and unbranched alkenyl groups, each optionally partially or fully halogenated. Examples of “alkenyl” include alkenyl groups that are straight chain alkenyl groups containing from two to ten carbon atoms and branched alkenyl groups containing from three to ten carbon atoms. Other examples include alkenyl groups which are straight chain alkenyl groups containing from two to six carbon atoms and branched alkenyl groups containing from three to six carbon atoms. Alkenyl groups include but are not limited to ethenyl, propenyl, n-butenyl, isobutenyl, 3-methylbut-2-enyl, n-pentenyl, heptenyl, octenyl, decenyl, and the like.

The term “alkynyl” refers to a mono or polyunsaturated aliphatic hydrocarbon radical that contains at least one carbon-carbon triple bond. “Alkynyl” refers to both branched and unbranched alkynyl groups, each optionally partially or fully halogenated. Examples of “alkynyl” include alkynyl groups that are straight chain alkynyl groups containing from two to eight carbon atoms and branched alkynyl groups containing from four to eight carbon atoms. Other examples include alkynyl groups that are straight chain alkynyl groups containing from two to six carbon atoms and branched alkynyl groups containing from four to six carbon atoms. This term is exemplified by groups such as ethynyl, propynyl, octynyl, and the like.

The term “cycloalkyl” refers to the mono- or polycyclic fused analogs of an alkyl group, as defined above. Unless otherwise specified, the cycloalkyl ring may be attached at any carbon atom that results in a stable structure and, if substituted, may be substituted at any suitable carbon atom which results in a stable structure. Examples of cycloalkyl groups are saturated cycloalkyl groups containing from three to ten carbon atoms. Other examples include cycloalkyl groups containing three to eight carbon atoms or three to six carbon atoms. It should be understood that any combination term using an “cycloalk” or “cycloalkylalkyl” refers to analogs according to the above definition of “cycloalkyl” including the number of carbon atoms. Exemplary cycloalkyl groups include but are not limited to cyclopropyl, cyclobutyl, cyclopentyl, cyclohexyl, cyclooctyl, cyclononyl, cyclodecyl, norbornyl, adamantyl, and the like.

The term “cycloalkenyl” refers to the mono- or polycyclic analogs of an alkenyl group, as defined above. Unless otherwise specified, the cycloalkenyl ring may be attached at any carbon atom that results in a stable structure and, if substituted, may be substituted at any suitable carbon atom which results in a stable structure. Examples of cycloalkenyl groups are cycloalkenyl groups containing from four to ten carbon atoms. Other examples include cycloalkenyl groups containing four to eight carbon atoms or four to six carbon atoms. Exemplary cycloalkenyl groups include but are not limited to cyclobutenyl, cyclopentenyl, cyclohexenyl, norbornenyl, and the like.

The term “heterocycloalkyl” refers to the mono- or polycyclic structures of “cycloalkyl” where one or more of the carbon atoms are replaced by one or more atoms independently selected from nitrogen, oxygen, or sulfur atoms. Any nitrogen atom maybe optionally oxidized or quaternized, and any sulfur atom maybe optionally oxidized. Generally, the heteroatoms may be selected from, but are not limited to, the group consisting of N, S, S═O, S(═O)₂, and O. Unless otherwise specified, the heterocycloalkyl ring may be attached at any carbon atom or heteroatom that results in a stable structure and, if substituted, may be substituted at any suitable carbon atom or heteroatom which results in a stable structure. Examples of heterocycloalkyl groups are saturated heterocycloalkyl groups containing from two to nine carbon atoms and one to four heteroatoms. Generally, 5-7 membered heterocycloalkyl groups contain 3-6 carbon atoms and 1-2 heteroatoms independently selected from the group consisting of N, S, S═O, S(═O)₂, and O. Examples of heterocycloalkyl groups include but are not limited to morpholino, pyrazino, tetrahydrofurano, and the like. “Carbon-attached heterocycloalkyl” refers to a heterocycloalkyl group which is bound via a constituent carbon atom. A heterocycloalkyl that is fused with a phenyl can include, but is not limited to, the following:

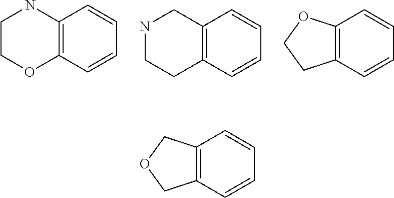

A heterocycloalkyl that is fused with a 5-6 membered heteroaryl can include, but is not limited to, the following:

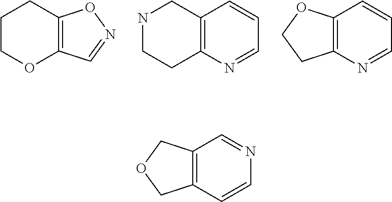

The term “heterocycloalkenyl” refers to the mono- or polycyclic structures of “cycloalkenyl” where one or more of the carbon atoms are replaced by one or more atoms independently chosen from nitrogen, oxygen, or sulfur atoms. Any nitrogen atom maybe optionally oxidized or quaternized, and any sulfur atom maybe optionally oxidized. Unless otherwise specified, the heterocycloalkenyl ring may be attached at any carbon atom or heteroatom that results in a stable structure and, if substituted, may be substituted at any suitable carbon atom or heteroatom which results in a stable structure. Examples of heterocycloalkenyl groups are heterocycloalkenyl groups containing from two to nine carbon atoms and one to four heteroatoms. Generally, 5-7 membered heterocycloalkenyl groups contain 3-6 carbon atoms and 1-2 heteroatoms independently selected from the group consisting of N, S, S═O, S(═O)₂, and O. Examples of heterocycloalkenyl groups include but are not limited to dihydropyran, dihydrofuran, and the like. “Carbon-attached heterocycloalkenyl” refers to a heterocycloalkenyl group which is bound via a constituent carbon atom.

The term “cycloalkyloxy” refers to a monovalent radical of the formula —O-cycloalkyl, i.e., a cycloalkyl group linked to a second group via an oxygen atom, wherein the cycloalkyl group is as defined above including optionally substituted cycloalkyl groups as also defined herein.

The term “acyl” refers to a monovalent radical of the formula —C(═O)-alkyl and —C(═O)-cycloalkyl, i.e., an alkyl or cycloalkyl group as defined above linked to a second group via carbonyl group C(═O), wherein said alkyl maybe further substituted with cycloalkyl, aryl, or heteroaryl. Examples of acyl groups include —C(═O)Me (acetyl), —C(═O)CH₂-cyclopropyl (cyclopropylacetyl), —C(═O)CH₂Ph (phenylacetyl), and the like.

The term “aryl” refers to 6-10 membered mono- or polycyclic aromatic carbocycles, for example, phenyl and naphthyl. Unless otherwise specified, the aryl ring may be attached at any carbon atom that results in a stable structure and, if substituted, may be substituted at any suitable carbon atom which results in a stable structure. The term “aryl” refers to non-substituted aryls and aryls optionally substituted with one or more substituents. Aryl maybe abbreviated “Ar”. It should be understood that any combination term using an “ar” or “aryl” prefix refers to analogs according to the above definition of “aryl” including the number of atoms. For example, terms such as “aryloxy”, “arylthio”, and “arylamino” refer to aryl groups linked to a second group via an oxygen, sulfur, or nitrogen atom, respectively.

The term “heteroaryl” refers to a stable 5-8 membered monocyclic or 8-11 membered bicyclic aromatic heterocycle radical. In one embodiment the monocyclic groups are 5 or 6 membered. Each heteroaryl contains 1-10 carbon atoms and from 1 to 5 heteroatoms independently chosen from nitrogen, oxygen and sulfur, wherein any sulfur heteroatom may optionally be oxidized and any nitrogen heteroatom may optionally be oxidized or quaternized. Unless otherwise specified, the heteroaryl ring may be attached at any suitable heteroatom or carbon atom that results in a stable structure and, if substituted, may be substituted at any suitable heteroatom or carbon atom which results in a stable structure. The term “heteroaryl” includes heteroaryl groups that are non-substituted or those optionally substituted. Generally, heteroaryl groups containing 2-9 carbon atoms and 1-4 heteroatoms independently selected from the group N, S, S═O, S(═O)₂, and O. Examples of “heteroaryl” include but are not limited to radicals such as furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzofuranyl, benzothienyl, indazolyl, benzimidazolyl, benzothiazolyl, benzoxazolyl, benzisoxazolyl, benzisothiazolyl, purinyl, quinolizinyl, quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl and phenoxazinyl. Terms such as “heteroaryloxy”, “heteroarylthio”, “heteroarylamino” refer to heteroaryl groups linked to a second group via an oxygen, sulfur, or nitrogen atom, respectively.

Each of the groups described herein, including alkyl, haloalkyl, alkenyl, alkynyl, cycloalkyl, cycloalkenyl, heterocycloalkyl, heterocycloalkenyl, cycloalkyloxy, acyl, aryl, heteroaryl, all are optionally substituted.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution. A group may have 1-6, or 1-3, or 1-2 optional substituents. Exemplary optional substituents include one or more of the following groups: halogen, C₁-C₆ alkyl, C₃-C₆ cycloalkyl, C₁-C₆ haloalkyl C₂-C₆ alkenyl, C₄-C₆ cycloalkenyl, C₂-C₆ alkynyl, nitro, nitrile, cyano, hydroxyl, C₁-C₆ alkoxy, C₁-C₆ haloalkoxy, C₃-C₆ cycloalkoxy, amino, C₁-C₆ alkylamino (for example, —NHMe- or —N(Me)₂), C₁-C₆ acyl, thiol, alkylthio, and carboxylic acid. Additional optional substituents include aryl, heteroaryl, heterocycloalkyl and heterocycloalkenyl. Such substituents can further be substituted with optionally selected groups to form a stable structure.

“Isomers” mean any compound with an identical molecular formula but having a difference in the nature or sequence of bonding or arrangement of the atoms in space. Examples of such isomers include, for example, E and Z isomers of double bonds, enantiomers, and diastereomers.

The term “therapeutically effective amount” refers to an amount that has any beneficial effect in treating a disease or condition.

The term “pharmaceutically acceptable salt” includes salts of compounds of Formulae I-XVIII derived from the combination of a compound of this invention and an organic or inorganic acid or base. Suitable acids include HCl, HBr, sulfuric acid, acetic acid, phosphoric acid, oxalic acid, etc.

It should be noted that the disclosure makes reference to particular carbon atoms in the structures of the various disclosed formulas by referring to them by number, for example, C1, C2, C3, etc. The numbering of carbons in steroids and steroid-like structures is known in the art, and has been explained, for example, in Moss G. P. Nomenclature of Steroids, Pure & Appl. Chem., 61 (10) 1783-1822 (1989), which is hereby incorporated by reference in its entirety.

### Formulations

Compounds of the invention may be administered orally in a total daily dose of about 0.01 mg/kg/dose to about 100 mg/kg/dose, typically from about 0.1 mg/kg/dose to about 10 mg/kg/dose. The use of time-release preparations to control the rate of release of the active ingredient may be employed. The dose may be administered in as many divided doses as is convenient. When other methods are used (e.g. intravenous administration) compounds may be administered at a rate from 0.05 to 10 mg/kg/hour, typically from 0.1 to 1 mg/kg/hour. Such rates are easily maintained when these compounds are intravenously administered as discussed below.

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters. Oral administration is generally employed.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax maybe employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative,- a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions. The pharmaceutical composition can be prepared to provide easily measurable amounts for administration. For example, an aqueous solution intended for intravenous infusion should contain from about 3 to 330 g of the active ingredient per milliliter of solution in order that infusion of a suitable volume at a rate of about 30 mL/hr can occur.

As noted above, formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of formula I when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

Suitable unit dosage formulations are those containing a daily dose or unit, daily sub-dose, or an appropriate fraction thereof, of a compound of Formula I.

It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

The following synthetic schemes are representative examples of the synthesis of compounds of Formulae II-XVIII. One of skill would understand how to apply these representative synthetic schemes to synthesis of compounds of Formulae II-XVIII.

The isoxazoles of Formula IIa were prepared from 20-keto steroids as shown in Scheme 1. Haloform reaction formed the 17β-carboxylic acid, which was reduced to the alcohol and then oxidized to the 17β-aldehyde. Reaction of the aldehyde with hydroxylamine gave the oxime which was oxidized with N-chlorosuccinimide (NCS) and added to the desired alkyne (HC—CR₁₉).

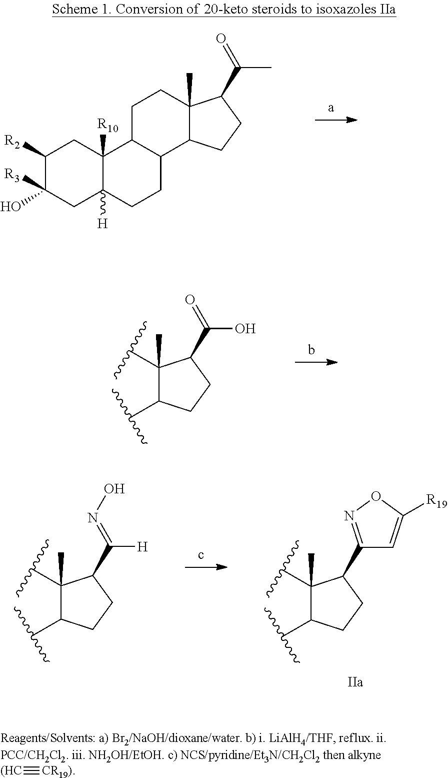

17β-Isoxazoles of Formula II can also be prepared via 20-ketoximes as shown in Scheme 2 (Nitz, T. J. et al. J. Org. Chem. 1994, 59, 5828-5832). The 20-oxime is converted to a dianion with nBuLi and then condensed with the desired ester. Addition of acid then affords the oxime II after deprotection to the free 3α-ol.

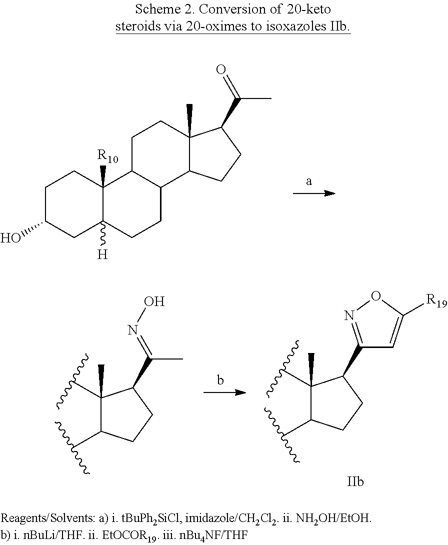

The synthesis of 17β-isoxazoles of Formula IIIa from 20-keto steroids is given in Scheme 3 below. The reaction to form the adduct with diethyloxalate was performed as described in U.S. Pat. Nos. 2,683,724 and 2,740,783. Addition of hydroxylamine then gave the expected isoxazole ester which was reduced to the 3-hydroxymethylisoxazole (Nazare, et al. J. Med. Chem. 2005, 48, 4511-4525). The unsubstituted isoxazoles IIc and IIIb (R₁₉=H) were prepared as described by Doorenbos, et al. J. Org. Chem. 1966, 31, 3193-3199 from 21-formyl-20-ketosteroids.

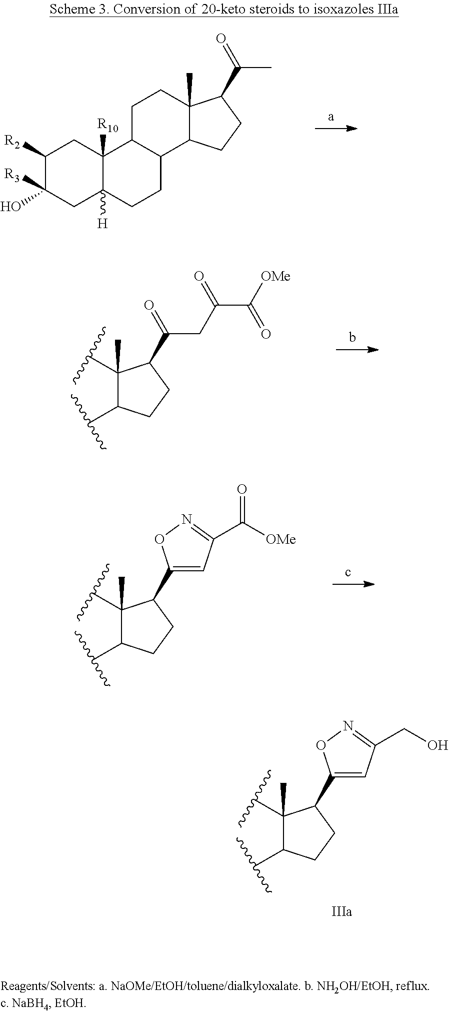

Isoxazoles of Formula IIIc can also be prepared from 17β-ethynyl steroids (Scheme 4; Souli, et al. J. Med. Chem. 2005, 48, 5203-5214). The 17β-ethynyl steroids can also be prepared directly from the 20-keto steroids via the vinyl triflate (Perez-Garcia, X. et al. Org. Lett. 2003, 5, 4033-4036).

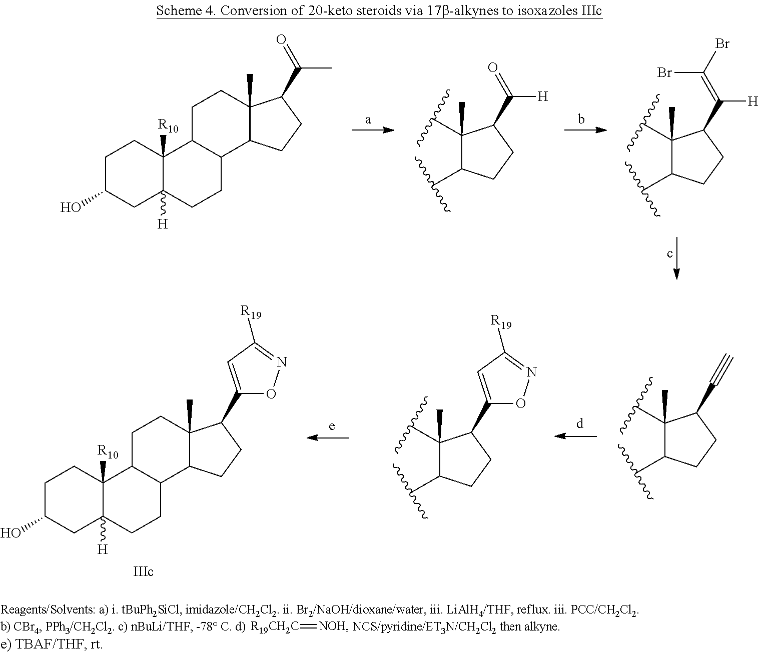

17β-isoxazol-5-yl steroids can be synthesized from 21-bromo-20-keto steroids by reaction with acetamides in a sealed tube (Scheme 5, see for example Kim, et al. WO 2010/046780). Reaction of the 21-bromide with 2-aminopyridine affords the expected 2-substituted-imidazo[1,2-a]pyridine (Catsoulacos and Souli J. Heterocyclic Chem. 1974, 11, 87). The isomeric 17β-isoxazol-2-yl steroids are prepared as described in LaMattina et al. J. Org. Chem. 1980, 45, 2261 starting with 21-amino-20-keto steroids and reaction with a trialkylorthoformate. They can also be synthesized by oxidizing the corresponding oxazines with MnO₂. The oxazines can be prepared from 17β-N-(2-hydroxyethyl)carboxamides with TsCl/pyr or with Burgess reagent. (see Scheme 6 and WO 2010/046780 and Rasmusson et al. J. Med. Chem. 1986, 29, 2298). 17β-oxazol-2-yl-substituted steroids have also been prepared as described by Zhu et al. Steroids 2003, 68, 603-611 (Scheme 6).

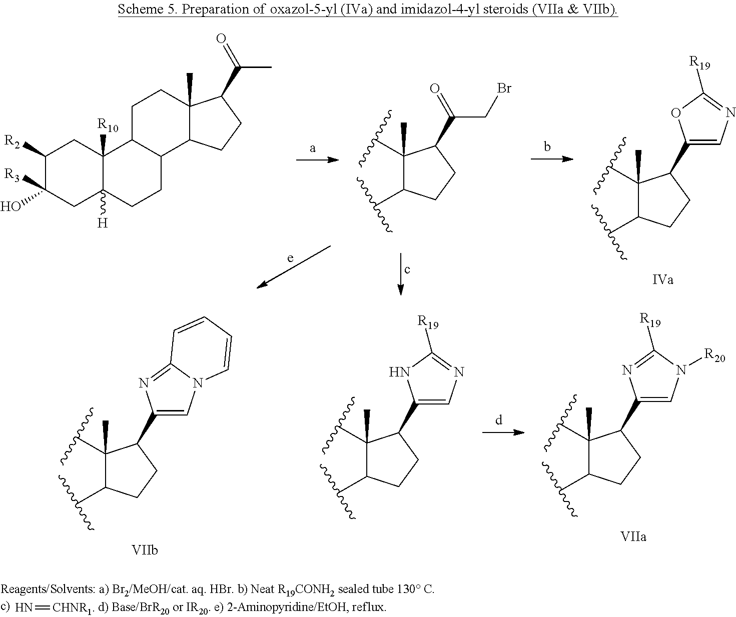

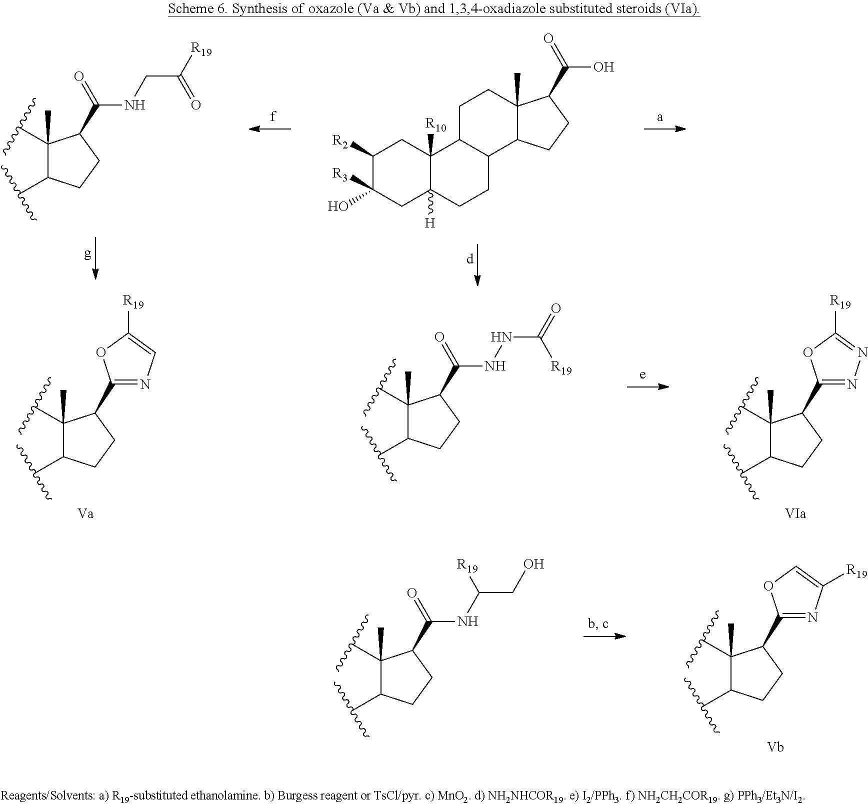

Steroids substituted with N-attached azoles can be prepared from 17-ketosteroids as shown in Scheme 7. Conversion of the 3α-hydroxyl to the corresponding acetate, followed by reaction with POCl₃ and N-formylmorpholine or DMF affords the desired β-chloro-α,β-unsaturated aldehyde. (Sciaky and Pallini Tetrahedron Lett. 1964, 28, 1839; Dalmaris et al. J. Chem. Research (S) 2003, 150 and Njar et al. J. Med. Chem. 1998, 41, 902). Addition of azoles then gives the expected substitution product (Handratta, et al. J. Med. Chem. 2005, 48, 2972; Handratta, et al. Njar, et al. J. Med. Chem. 1998, 41, 902; Njar et al. Bioorg. Med. Chem. Lett. 1996, 6, 2777) which is deformylated with Wilkinson's catalyst and hydrogenated with hydrazine in air.

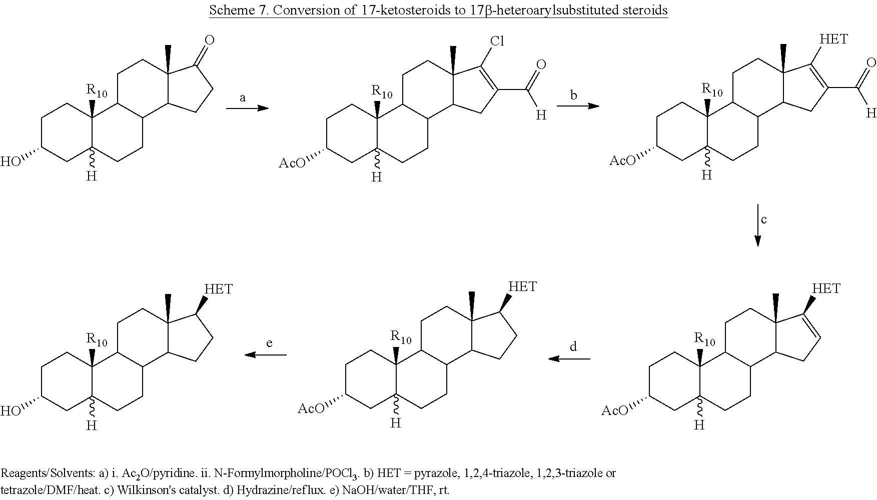

These azoles can also be prepared from the corresponding vinyl triflate (Steroids 2010, 75 936-943) as in Scheme 8.

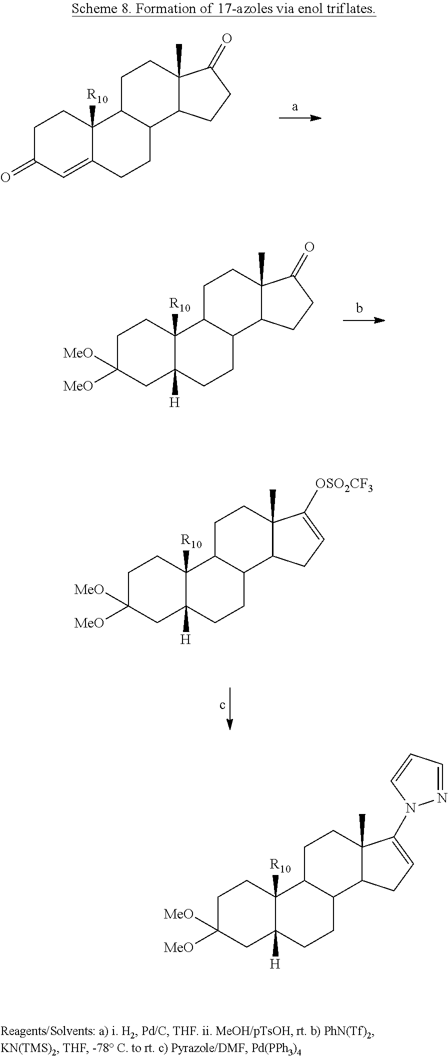

3-Steroid substituted-1,2,4-oxadiazoles can be prepared as described in WO 2009/085433. The isomeric 5-steroid substituted-1,2,4-oxadiazoles can be prepared as described by Gangloff et al. Tetrahedron Lett. 2001, 42, 1441 and Chiou and Shine J. Heterocyclic Chem. 1989, 26, 125. When Het=2-pyridyl, the 17-one can be converted to a vinyl iodide and coupled with a boronic acid (Steroids 2006, 71, 585-590) or 2-pyridyl trialkylstannane (Scheme 9). The corresponding pyrazine is prepared by coupling the iodide with (2-tributylstanyl)pyrazine (Handratta, et al. J. Med. Chem. 2005, 48, 2972). The 16-ene is then reduced and the 3-ketal is transformed into the desired 3α-ol.

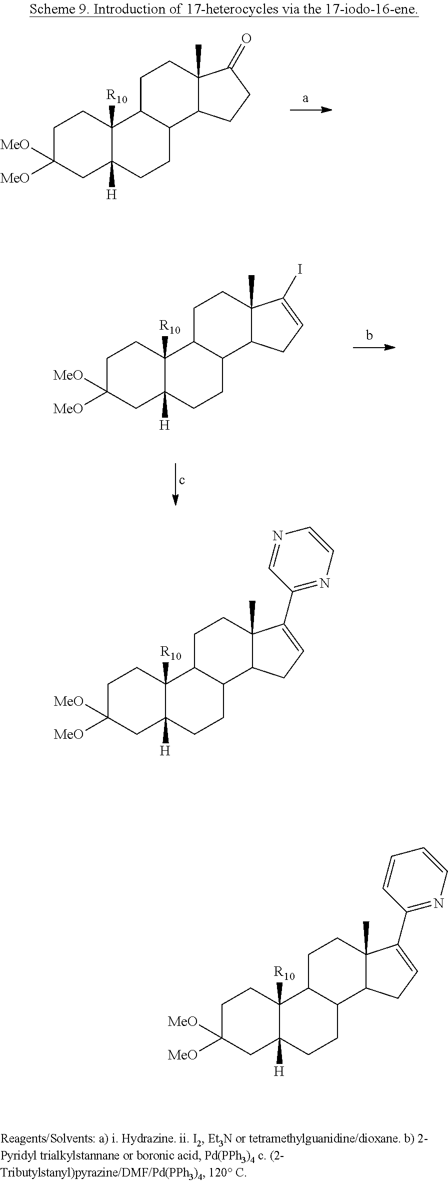

The synthesis of the pyrimidine XIVa can also be accomplished as shown in Scheme 10 starting from the enaminone derived from a 20-keto steroid. Reaction with an amidate or guanidine affords the substituted pyrimidine. See for example WO 2009/057079 and 109907 and J. Het. Chem. 2010, 47, 887. Reaction of the intermediate enaminone with an acylhydrazide forms the corresponding pyridazine XVa (Mohareb, R. et al. Acta Pharmaceutica 2008, 58(1), 29).

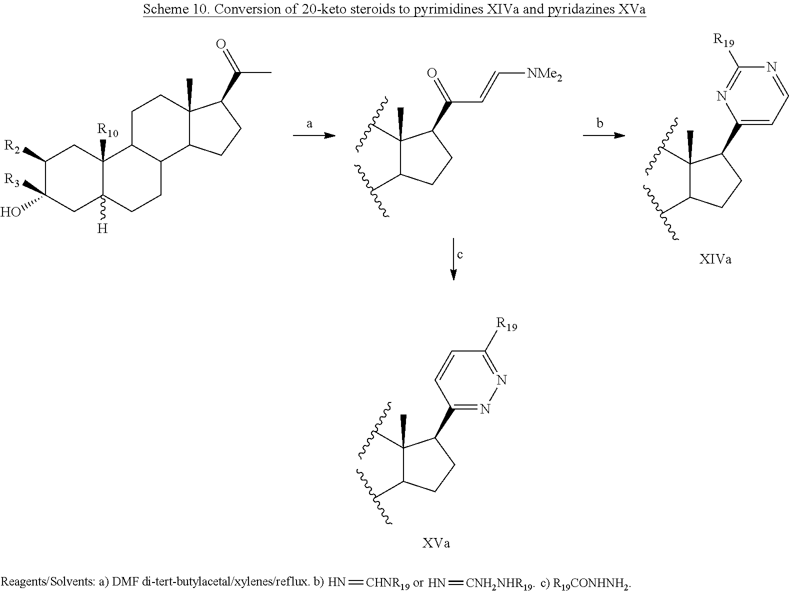

The isomeric pyrimidine XIIIa can be prepared as shown in Scheme 11. The 17β-nitrile (Han, et al. J. Med. Chem. 1996, 39, 4218) is converted to the amidine and then to XIIIa as described in WO 2009/132000 and WO 2009/012275. Conversion of the nitrile to the imidate followed by reaction as described in Bach, A. et al. J. Heterocyclic Chem. 2004, 41, 637, affords the 1,2,4-triazene XVIIIa. The isomeric 1,2,4-triazene XIIIb can be prepared from the 21-hydroxy-20-keto steroid by reaction with a substituted acetamidrazone using the procedure of Laphookpieo et al. Tetrahedron Lett. 2006, 47, 3865 as shown in Scheme 12.

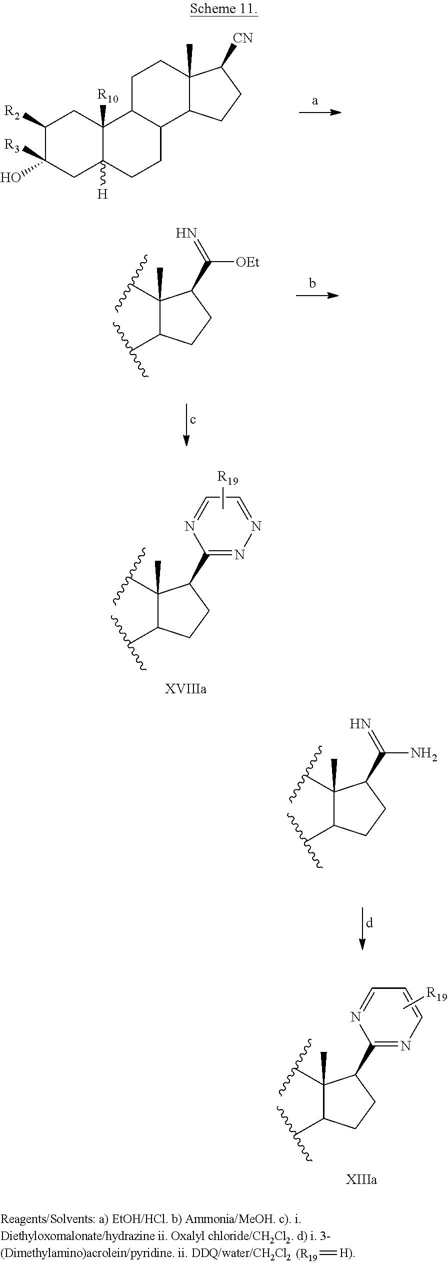

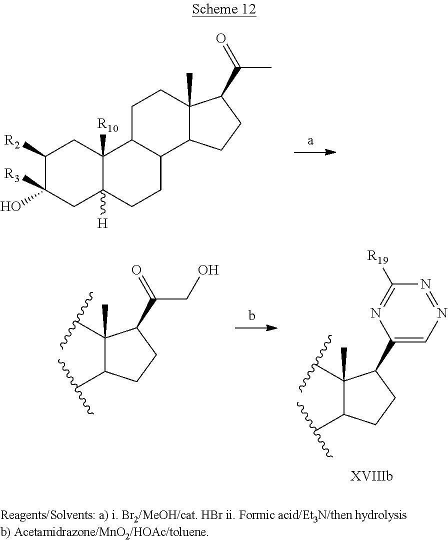

### Examples

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

### Example 1

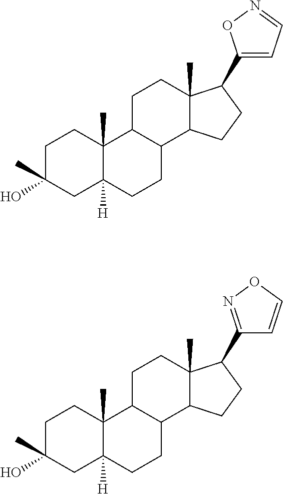 [SMILES: C[C@@]1(O)CC[C@]2(C)C3CC[C@@]4(C)C(CC[C@@H]4c4ccno4)C3CC[C@H]2C1 ; C[C@@]1(O)CC[C@]2(C)C3CC[C@@]4(C)C(CC[C@@H]4c4ccon4)C3CC[C@H]2C1]

### 5-[3α-hydroxy-3β-methyl-5α-androstan-17β-yl]-isoxazole and 3-[3α-hydroxy-3β-methyl-5α-androstan-17β-yl]-isoxazole

The title compounds were prepared as described by Doorenbos, et al. J. Org. Chem. 1966, 31, 3193-3199 starting with 3α-hydroxy-3β-methyl-5α-pregnan-20-one. The isomeric isoxazoles were separated by preparative RPHPLC using acetonitrile/water as eluent. TOF MS m/z 358 (M+H⁺).

### Example 2

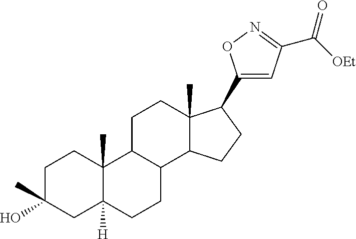 [SMILES: CCOC(=O)c1cc([C@H]2CCC3C4CC[C@H]5C[C@](C)(O)CC[C@]5(C)C4CC[C@@]32C)on1]

### Ethyl 5-[3α-hydroxy-3β-methyl-5α-androstan-17β-yl]-3-isoxazolecarboxylate

### Ethyl 20,23-dioxo-3α-hydroxy-3β-methyl-21-norcholanoate

A 250 mL flask was charged with 5 mL of dry EtOH and 168 mg of sodium metal was added. Once the sodium had reacted, 8 mL of dry toluene was added, followed by 0.8 mL of diethyl oxalate. A solution of 3α-hydroxy-3β-methyl-5α-pregnan-20-one (2.0 g) in 5 mL of EtOH and 25 mL of toluene was added in a slow stream to the reaction at 0° C. The cold bath was removed and the reaction was stirred for 2 h 40 m. The reaction was diluted with 200 mL of ether and allowed to stand at rt. The ppt that formed was isolated and washed with ether to give 700 mg of the title compound as an off-white solid.

### Ethyl 5-[3α-hydroxy-3β-methyl-5α-androstan-17β-yl]-3-isoxazolecarboxylate

A suspension of the adduct above (234 mg, 0.541 mmol) in 2 mL of dry EtOH was treated with hydroxylamine hydrochloride (134 mg) and heated at reflux for 5 h. Once at rt, the reaction was conc. to dryness and partitioned between EtOAc and a sat. aq. NaHCO₃ solution. The organic layer was separated, washed with brine, dried (MgSO₄), filtered and conc. Purification by RPHPLC gave 96 mg of the isoxazole as a white solid. MS m/z 452 (M+Na⁺).

### Example 3

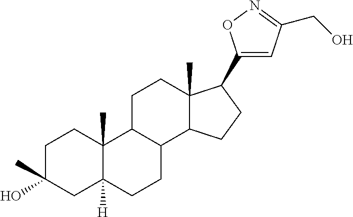 [SMILES: C[C@@]1(O)CC[C@]2(C)C3CC[C@@]4(C)C(CC[C@@H]4c4cc(CO)no4)C3CC[C@H]2C1]

### 5-[3α-hydroxy-3β-methyl-5α-androstan-17β-yl]-3-hydroxymethylisoxazole

A solution of ethyl 5-[3α-hydroxy-3β-methyl-5α-androstan-17β-yl]-3-isoxazolecarboxylate (32 mg, 0.074 mmol) in 1 mL of dry EtOH cooled in an ice/water bath was treated with solid NaBH₄ (40 mg). After stirring overnight at rt, the reaction was added to ice/water and extracted with EtOAc. The aqueous layer was extracted twice with additional EtAOc. The pooled EtOAc layers were washed with brine, dried (MgSO₄), filtered and concentrated. Flash column chromatography (5% MeOH/CH₂Cl₂) gave 22 mg of the title compound as a white solid, mp 192-193° C. MS m/z 388 (M+H⁺).

### Example 4

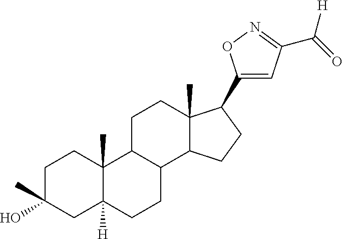 [SMILES: C[C@@]1(O)CC[C@]2(C)C3CC[C@@]4(C)C(CC[C@@H]4c4cc(C=O)no4)C3CC[C@H]2C1]

### 5-[3α-hydroxy-3β-methyl-5α-androstan-17β-yl]-3-isoxazolecarboxaldehyde

A solution of 5-[3α-hydroxy-3β-methyl-5α-androstan-17β-yl]-3-hydroxymethylisoxazole (17 mg) in 2 mL of CH₂Cl₂ was treated with 41 mg of NaOAc and solid PCC (48 mg) was added. After stirring overnight at rt, the reaction was added directly to a flash silica gel column. Elution with 100% CH₂Cl₂ and 9:1 CH₂Cl₂/EtOAc gave 7 mg of the aldehyde.

### Example 5

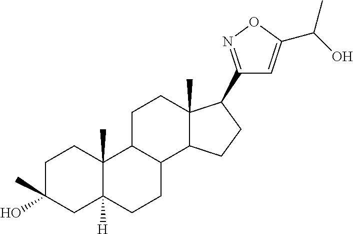 [SMILES: CC(O)c1cc([C@H]2CCC3C4CC[C@H]5C[C@](C)(O)CC[C@]5(C)C4CC[C@@]32C)no1]

### (S)-3-[3α-hydroxy-3β-methyl-5α-androstan-17β-yl]-5-(1-hydroxyethyl)isoxazole 3α-Hydroxy-3β-methyl-5α-androstane-17β-carboxylic acid

The acid was prepared from 3α-hydroxy-3β-methyl-5α-pregnan-20-one as described by Gee, et al. WO 1994/27608.

### 3α-Hydroxy-17β-hydroxymethyl-3β-methyl-5α-androstane

3α-Hydroxy-3β-methyl-5α-androstane-17β-carboxylic acid (0.34 g, 1.00 mmol) in 10 mL of THF was cooled in a water bath and treated with 1 mL (1.00 mmol) of a 1M solution of LiAlH₄ in THF added dropwise. The reaction was heated at reflux for 4 hr and allowed to cool to rt. The reaction was partitioned between EtOAc and a 1M aq. HCl solution. The organic layer was separated, dried with Na₂SO₄, filtered and conc. to give the alcohol as a white solid.

### 3α-Hydroxy-3β-methyl-5α-androstan-17β-carboxaldehyde

A solution of 3α-hydroxy-17β-hydroxymethyl-3β-methyl-5α-androstane (0.40 g, 1.25 mmol) in 15 mL of CH₂Cl₂ was treated with 1.2 g of Celite and 0.39 g of PCC. After stirring at rt for 6 h, the reaction was poured onto a flash silica column. Elution with 100% CH₂Cl₂ gave the aldehyde as a yellow solid.

### 3α-Hydroxy-3β-methyl-5α-androstan-17β-carboxaldehyde oxime

A solution of 3α-hydroxy-3β-methyl-5α-androstan-17β-carboxaldehyde (280 mg, 0.88 mmol) in 10 mL water and 2 mL EtOH was treated with 0.10 g (1.3 mmol) of hydroxylamine hydrochloride and 178 mg (1.3 mmol) of sodium carbonate. After stirring at rt for 4 h, the reaction was concentrated to dryness and washed with cold water, affording the oxime as a white solid.

MS m/z 334 (M+H⁺).

### (S)-3-[3α-hydroxy-3β-methyl-5α-androstan-17β-yl]-5-(1-hydroxyethyl)isoxazole

A solution 3α-hydroxy-3β-methyl-5α-androstan-17β-carboxaldehyde oxime (81 mg, 0.24 mmol) in 2 mL of CH₂Cl₂ was treated with 2 drops of pyridine and 33 mg of N-chlorosuccinimide. After stirring for 50 min, the reaction was treated with neat (S)-3-butyn-2-ol (100 μL, 88 mg, 1.3 mmol) followed by neat N,N-diisopropylethylamine (45 μL). After stirring overnight, the reaction was cone in vacuo. The residue was absorbed onto silica gel and subjected to flash chromatography. Elution with 7:3 EtOAc/hexanes gave 15 mg of the alcohol as a solid. TOF MS m/z 402 (M+H⁺).

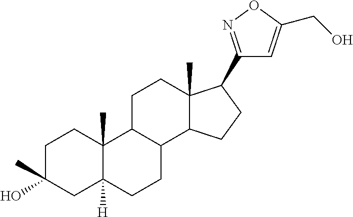 [SMILES: C[C@@]1(O)CC[C@]2(C)C3CC[C@@]4(C)C(CC[C@@H]4c4cc(CO)on4)C3CC[C@H]2C1]

### 3-[3α-hydroxy-3β-methyl-5α-androstan-17β-yl]-5-(hydroxymethyl)isoxazole

3-[3α-Hydroxy-3β-methyl-5α-androstan-17β-yl]-5-(hydroxymethyl)isoxazole was prepared as described above for (S)-3-[3α-hydroxy-3β-methyl-5α-androstan-17β-yl]-5-(1-hydroxyethyl)isoxazole except that (S)-3-butyn-2-ol was replaced with propyn-3-ol. The compound exhibited mp 198.5-200° C.

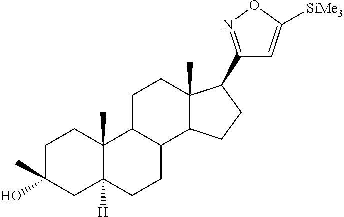 [SMILES: C[C@@]1(O)CC[C@]2(C)C3CC[C@@]4(C)C(CC[C@@H]4c4cc([Si](C)(C)C)on4)C3CC[C@H]2C1]

### 3-[3α-hydroxy-3β-methyl-5α-androstan-17β-yl]-5-(trimethylsilyl)isoxazole

3-[3α-Hydroxy-3β-methyl-5α-androstan-17β-yl]-5-(trimethylsilyl)isoxazole was prepared as described above for (S)-3-[3α-hydroxy-3β-methyl-5α-androstan-17β-yl]-5-(1-hydroxyethyl)isoxazole except that (S)-3-butyn-2-ol was replaced with trimethylsilylacetylene. TOF MS m/z 430 (M+H⁺).

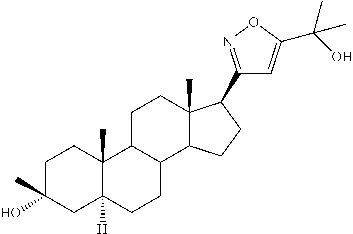 [SMILES: CC(C)(O)c1cc([C@H]2CCC3C4CC[C@H]5C[C@](C)(O)CC[C@]5(C)C4CC[C@@]32C)no1]

### 3-[3α-Hydroxy-3β-methyl-5α-androstan-17β-yl]-5-(2-hydroxy-2-propyl)isoxazole

3-[3α-Hydroxy-3β-methyl-5α-androstan-17β-yl]-5-(2-hydroxy-2-propyl)isoxazole was prepared by using the method described for 3-[3α-hydroxy-3β-methyl-5α-androstan-17β-yl]-5-(hydroxymethyl)isoxazole except that propargyl alcohol was replaced with 2-methyl-3-butyn-2-ol. TOF MS m/z 416 (M+H⁺).

### Example 6

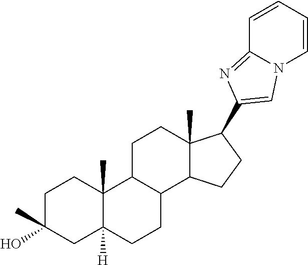 [SMILES: C[C@@]1(O)CC[C@]2(C)C3CC[C@@]4(C)C(CC[C@@H]4c4cn5ccccc5n4)C3CC[C@H]2C1]

### 2-[3α-Hydroxy-3β-methyl-5α-androstan-17β-yl]-imidazo[1,2-a]pyridine

21-Bromo-3α-hydroxy-3β-methyl-5α-pregnan-20-one. A solution of 3α-hydroxy-3β-methyl-5α-pregnan-20-one (5.05 g, 15.2 mmol) in 225 mL of MeOH was treated with 2 drops of a 48% aq. HBr solution and a solution of Br₂ (2.68 g, 16.7 mmol) in 10 mL of MeOH was added dropwise over 25 min. The resulting ppt was collected, affording 4.68 g of crude product. Recrystallization from 75 mL of toluene gave 3.76 g of the 21-bromide as a white solid.

### 2-[3α-Hydroxy-3β-methyl-5α-androstan-17β-yl]-imidazo[1,2-a]pyridine

A mixture of 21-bromo-3α-hydroxy-3β-methyl-5α-pregnan-20-one (161 mg, 0.39 mmol) and 2-aminopyridine (38 mg, 0.39 mmol) in 2 mL of EtOH was refluxed for 5 hr. The EtOH was removed in vacuo and the residue was subjected to flash chromatography. Elution with 4% MeOH/CH₂Cl₂ gave 44 mg of the title compound as a white solid. Mp 225-226° C.

### Example 7

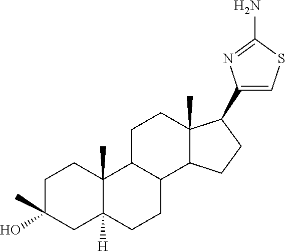 [SMILES: C[C@@]1(O)CC[C@]2(C)C3CC[C@@]4(C)C(CC[C@@H]4c4csc(N)n4)C3CC[C@H]2C1]

### 2-Amino-4-[3α-hydroxy-3β-methyl-5α-androstan-170-yl]-thiazole

A mixture of 21-bromo-3α-hydroxy-3β-methyl-5α-pregnan-20-one (222 mg, 0.54 mmol) and thiourea (44 mg, 0.58 mmol) in 3 mL of EtOH was refluxed for 1 hr. The EtOH was removed in vacuo and the residue was triturated with 1% MeOH/CH₂Cl₂ affording 193 mg of the title compound as a solid. Mp 232-233° C.

### Example 8

### In vitro activity of 2-[3α-hydroxy-3β-methyl-5α-androstan-17β-yl]-imidazo[1,2-a]pyridine

2-[3α-Hydroxy-3β-methyl-5α-androstan-17β-yl]-imidazo[1,2-a]pyridine was tested in the [³⁵S]TBPS binding assay as described below. The compound exhibited IC₅₀=1.0 μM with Iₘₐₓ=100%.

### Example 9

### In vitro activity of 5-[3α-hydroxy-3β-methyl-5α-androstan-17β-yl]-isoxazole

5-[3α-Hydroxy-3β-methyl-5α-androstan-17β-yl]-isoxazole was tested in oocytes expressing human α₁β₁γ₂ GABA_{A} receptors as described below. The compound showed maximum modulation of 400% with EC₅₀=0.3 M.

### Example 10

Anticonvulsant activity of 3-[3α-hydroxy-3β-methyl-5α-androstan-17β-yl]-5-(hydroxymethyl)isoxazole

3-[3α-Hydroxy-3β-methyl-5α-androstan-17β-yl]-5-(hydroxymethyl)isoxazole was tested for its ability to protect animals from pentylenetetrazole-induced seizures as described below and showed an ED₅₀ of 4 mg/kg after oral dosing in 20% 2-hydroxypropyl-β-cyclodextrin.

### Example 11

Activity of 3-[3α-hydroxy-3β-methyl-5α-androstan-17β-yl]-5-(hydroxymethyl)isoxazole in the elevated plus maze 3-[3α-Hydroxy-3β-methyl-5α-androstan-17β-yl]-5-(hydroxymethyl)isoxazole was tested in the elevated plus maze (EPM) as described below and exhibited a minimum effective oral dose of 1 mg/kg when dosed in 20% 2-hydroxypropyl-β-cyclodextrin.

### Example 12

### Activity of 3-[3α-hydroxy-3β-methyl-5α-androstan-17β-yl]-5-(hydroxymethyl)isoxazole in the mouse rotarod paradigm

3-[3α-Hydroxy-3β-methyl-5α-androstan-17β-yl]-5-(hydroxymethyl)isoxazole was tested in the mouse rotarod as described below and was found to have an oral AD₅₀ of 43 mg/kg when dosed in 20% 2-hydroxypropyl-β-cyclodextrin.

### [³⁵]TBPS Binding Assay.

The cerebral cortex from male Sprague-Dawley rats (weighing 160-200 g) was removed immediately after decapitation and dissected over ice. A P₂ homogenate was prepared for binding assay as previously described (Gee, 1987a). The tissue was homogenized in 0.32 M sucrose (J. T. Baker Chemical Co., Phillipsburg, N.J.) with a Teflon-coated pestle, followed by centrifugation at 1,000×g for 10 min. The supernatant was collected and centrifuged at 9,000×g for 20 min. The resultant P₂ pellet was resuspended in ice-cold 50 mM sodium potassium phosphate buffer (pH 7.4) containing 200 mM NaCl and used immediately in binding assays. A 2 nM concentration of [³⁵S]TBPS (86 Ci/mmol; New England Nuclear, Boston, Mass.) was incubated with 100 μL of tissue homogenate (10% w/v) in the presence or absence of 5 μM GABA (Sigma Chem. Co., St. Louis, Mo.) and 5 μL aliquots of test drug dissolved in dimethyl sulfoxide (Sigma Chem. Co.) (≦10 μL of solvent used in all assays). At the concentration (≦1%) used, dimethyl sulfoxide had no effect on specific [³⁵S]TBPS binding. All assays were brought to a final volume of 1 ml with 50 mM sodium potassium phosphate buffer (pH 7.4) containing 200 mM NaCl. Non-specific binding was defined as binding in the presence of 2 μM TBPS (NEN, Boston, Mass.) and accounted for ˜30% of the total binding. Assays were terminated after a 90 min steady-state incubation at 25° C. by rapid filtration through glass fiber filters (no. 32; Schleicher & Schuell, Keene, N.H.). Filter-bound radioactivity was quantified by liquid scintillation spectrophotometry. The data were evaluated by nonlinear regression (GraphPad, Inc., San Diego, Calif.) to obtain IC₅₀ (concentration at which half-maximal inhibition of radioligand occurs) and Iₘₐₓ (maximum percent inhibition) values. Compounds that inhibit [³⁵S]TBPS binding are known to be allosteric positive modulators of GABA_{A} receptors.

### Oocyte Electrophysiology

Preparation, micro-injection and maintenance of oocytes was as previously described (Ng et al., Proc. Natl. Acad. Sci., 2007, 104, 8059). Individual oocytes were injected with 0.005-50 ng of each subunit mRNA as follows (ratio of subunits in parentheses): GABA_{A} receptor subunit combinations (α_{1,2, or 3}, β_{1,2, or 3}, γ_{2L} or δ): (5:1:1). Stage IV-V oocytes were plucked from ovary membranes, defolliculized with collagenase Type IA (Worthington's) for 45 min and rinsed 10 times with Ringer's salt solution. cRNA was injected at 50 nL. Oocytes were tested 3-28 days after injection (n=3-7 per compound), in Ringer's salt solution by linear drug application method using electrodes with 1-2 mΩ tip resistance. Changes in membrane current were passed through a pre-amp, then through a T200 patch amplifier (Axon Instruments), with a bandpass filter 2 kHz. pClamp software was used to monitor, record, and analyze data. All compounds were tested with a 30 second pretreatment prior to co-application with EC₁₀ (concentration of GABA that evokes 10% of the maximum response) GABA for the control response. Responses in presence of test compound were calculated as % modulation above control. Concentration-response curves were fit to non-linear regression analysis on Prism 4.0 (GraphPad, San Diego, Calif.) for % maximal stimulation and EC₅₀. Compounds with activity in electrophysiological studies show functional activity as modulators of GABA_{A} receptors.

### Mouse Light-Dark (LD) Transition Model

Naïve mice are acclimated (1 hr) to a darkened room prior to administration of test compounds. Testing occurrs during peak brain levels of compounds, in automated LD boxes (Coulbourn Instruments, Whitehall, Pa.), tracked by infrared beam-breaking collar and TruScan software (Coulbourn Instruments). 400 lux light bulbs are placed 60 cm above the floor of the test box, centered on the light-half of the box. The time spent in the dark is recorded. Data are analyzed with GraphPad Prism 4.0 for statistical significance by one-way ANOVA with Dunnett's multiple comparison post-hoc test. Compounds that increase the amount of time the animals spend in the light have activity as anxiolytics.

### Mouse Elevated Plus Maze (EPM)

Mice were group housed and handled daily for 3 days prior to testing in the EPM (Coulbourn Instruments). Testing was conducted in a dimly lit room, with two 60 W bulbs pointed at the ceiling near the open arms (4 ft. above the maze). The maze was cleaned between each run. Automated counting of time spent in the open arms of the maze was achieved by using Med Associates (St. Albans, Vt.) MedPC-IV program. Data were analyzed with GraphPad Prism 4.0 for statistical significance by one-way ANOVA with Dunnett's multiple comparison post-hoc test. Compounds that increase the amount of time the animals spend in the open arms have activity as anxiolytics.

### Mouse Rotarod (RR)

Naïve mice were trained on a RR (Columbus Instruments, Columbus, Ohio) in 4 sessions (6-15 rpm) over 2 days to successfully complete the 2-min trial prior to final testing (6 rpm). On day 3, the mice were administered compound and tested over a period of 360 min at various intervals. The percentage of animals remaining on the RR throughout each 2-min trial was recorded. The results that coincided with the time of peak effect were analyzed by the method of Litchfield and Wilcoxon (Litchfield and Wilcoxon, J. Pharmacol. Exp. Ther., 1949, 96, 99) to determine the AD₅₀ (ataxic half-maximal dose where half of the mice fail the RR assay). Compounds that have activity in the rotarod assay can indicate CNS depressive activity and sedative activity.

### Anticonvulsant Assay

The ability of compounds to protect against pentylenetetrazole-induced seizures was carried out as described by Hogenkamp, et al. J. Med. Chem. 2007, 50, 3369. The dose that protected half the animals from seizures was determined as the ED₅₀.

1. A compound of Formula I: 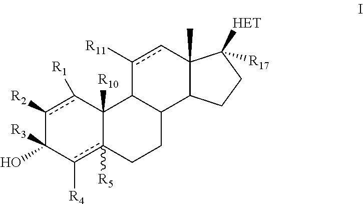
or a pharmaceutically acceptable salt, solvate, or prodrug thereof, wherein:
each R₁, R₂, R₃, R₄, and R₁₇ is independently selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁;
R₅ is a 5α or 5β-hydrogen, fluorine or absent if there is a C4-C5 double bond;
R₁₀ is hydrogen, fluorine or methyl;
R₁₁ is hydrogen, a hydroxyl, an NR₂₃R₂₄ group or a keto group;
each R₁₉ independently is hydrogen, halogen, C₁₋₈ alkyl, C₂₋₈ alkenyl, C₂₋₈ alkynyl, C₁₋₄ acyl, —C(═O)OC₁₋₄ alkyl, —C(═O)H, —Si(C₁₋₄ alkyl)₃, or C₁₋₈ haloalkyl, wherein each of said alkyl, alkenyl, alkynyl, and haloalkyl is optionally substituted with 1-5 R₂₁;
R₂₀ is selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁; or
R₂₀ is selected from the group consisting of aryl, heteroaryl, C₃₋₆ cycloalkyl, C₄₋₆ cycloalkenyl, heterocycloalkyl or heterocycloalkenyl, wherein said heterocycloalkyl is optionally fused with phenyl or a 5-6 membered heteroaryl having 1-3 heteroatoms, wherein one or more of the carbon atoms in said heterocycloalkyl or heterocycloalkenyl optionally may be oxidized to C(═O), wherein each of said aryl, heteroaryl, cycloalkyl, cycloalkenyl, heterocycloalkyl, and heterocycloalkenyl is optionally substituted with 1-5 R₂₂; or
R₁₉ and R₂₀ taken together with the atoms to which they are attached form a heteroaryl, a heterocycloalkyl or a heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), wherein each of said heteroaryl, heterocycloalkyl, and heterocycloalkenyl is optionally substituted with 1-5 R₂₂;
each R₂₁ is independently selected from the group consisting of hydroxyl, C₁₋₆ alkoxy, C₁₋₈ haloalkoxy, C₃₋₆ cycloalkoxy, NR₂₃R₂₄, aryl, heteroaryl, C₃₋₆ cycloalkyl, C₄₋₆ cycloalkenyl, heterocycloalkyl or heterocycloalkenyl, wherein each of said aryl, heteroaryl, cycloalkyl and cycloalkenyl are optionally substituted with 1-5 R₂₂; and wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), and wherein each of said heterocycloalkyl and said heterocycloalkenyl is optionally substituted with 1-5 R₂₂;
each R₂₂ is independently selected from the group consisting of nitro, nitrile, hydroxyl, halogen, C₁₋₆ acyl, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₆ cycloalkoxy, aryl, heteroaryl, —NR₂₃R₂₄, —C(═O)OR₂₃, —C(═O)NHR₂₃, —NHC(═O)R₂₅, —NHS(═O)₂R₂₅, —S(═O)₀₋₂R₂₅, —S(═O)₂NHR₂₃, C₃₋₆ cycloalkyl, C₄₋₆ cycloalkenyl, heterocycloalkyl or heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O);
each of R₂₃ and R₂₄ is independently hydrogen, C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted with 1-5 R₂₁;
R₂₅ is C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted;
HET is a heteroaryl group selected from 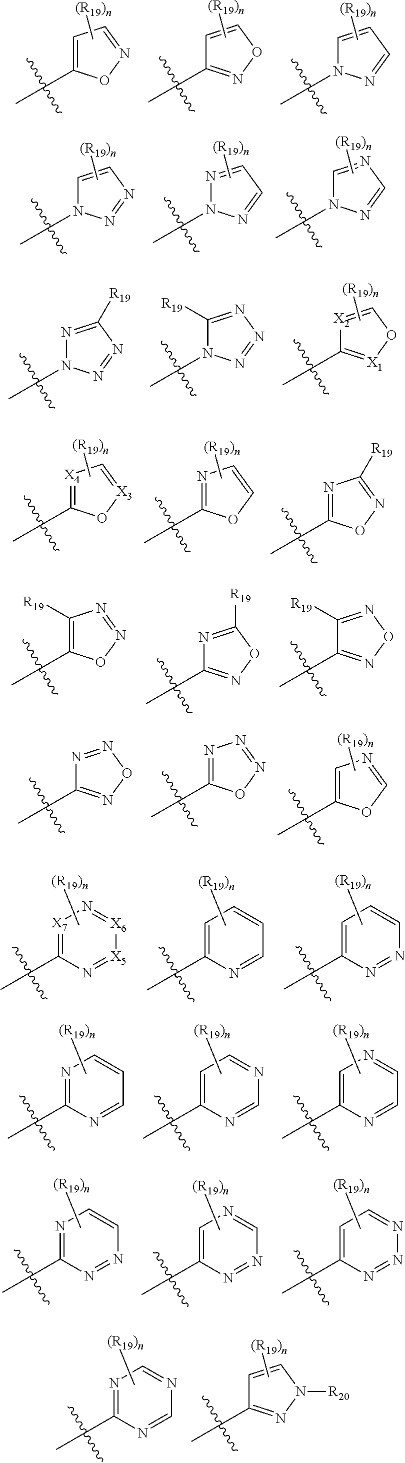 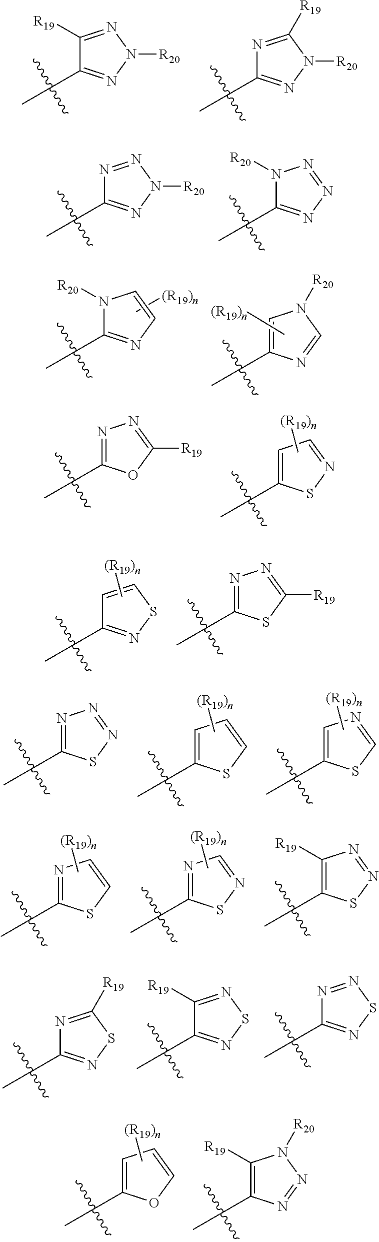
when X₁ is N, X₂ is CR₁₉ or N, when X₁ is CR₁₉, X₂ is N;
X₃ and X₄ are independently CR₁₉ and N;
when X₅ is N, X₆ and X₇ are independently CR₁₉; or
when X₆ is N, X₅ and X₇ are independently CR₁₉; or
when X₇ is N, X₅ and X₆ are independently CR₁₉;
n is an integer from 1 to 4;
the dashed lines represent optional double bonds; with the proviso that when the bond between C1 and C2 is a single bond, then R₂ and R₃ are not both hydrogen.

2. A compound of claim 1, wherein said compound is a compound of Formula II: 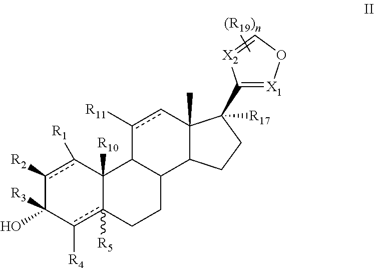
or a pharmaceutically acceptable salt, solvate, or prodrug thereof, wherein:
when X₁ is N, X₂ is CR₁₉ or N, when X₁ is CR₁₉, X₂ is N;
n is an integer from 1 to 2;
the dashed lines represent optional double bonds; with the proviso that when the bond between C1 and C2 is a single bond, then R₂ and R₃ are not both hydrogen.

3. A compound of claim 1, wherein said compound is a compound of Formula III: 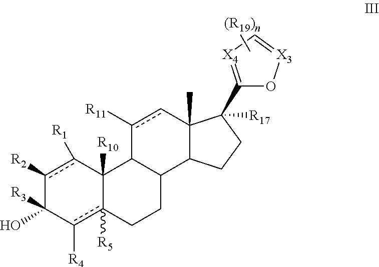
or a pharmaceutically acceptable salt, solvate, or prodrug thereof, wherein:
X₃ and X₄ are independently CR₁₉ or N;
n is an integer from 1 to 3
the dashed lines represent optional double bonds; with the proviso that when the bond between C1 and C2 is a single bond, then R₂ and R₃ are not both hydrogen.

4. A compound of claim 1, wherein said compound is a compound of Formula IV: 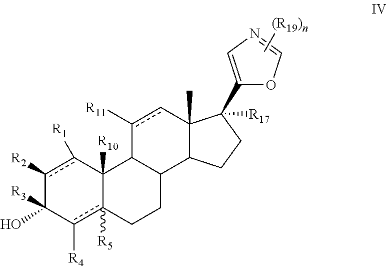
or a pharmaceutically acceptable salt, solvate, or prodrug thereof, wherein:
n is 1 or 2;
the dashed lines represent optional double bonds; with the proviso that when the bond between C1 and C2 is a single bond, then R₂ and R₃ are not both hydrogen.

5. A compound of claim 1, wherein said compound is a compound of Formula V: 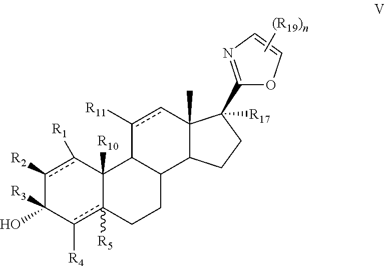
or a pharmaceutically acceptable salt, solvate, or prodrug thereof, wherein:
n is 1 or 2;
the dashed lines represent optional double bonds; with the proviso that when the bond between C1 and C2 is a single bond, then R₂ and R₃ are not both hydrogen.

6. A compound of claim 1, wherein said compound is a compound of Formula VI: 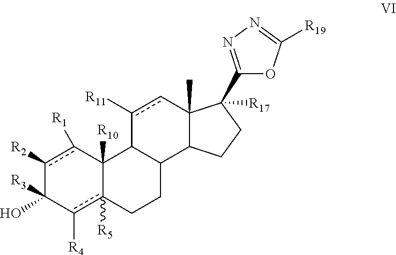
or a pharmaceutically acceptable salt, solvate, or prodrug thereof, wherein:
the dashed lines represent optional double bonds; with the proviso that when the bond between C1 and C2 is a single bond, then R₂ and R₃ are not both hydrogen.

7. A compound of claim 1, wherein said compound is a compound of Formula VII: 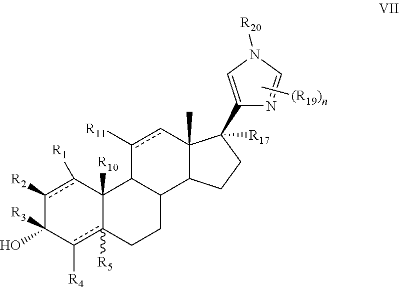
or a pharmaceutically acceptable salt, solvate, or prodrug thereof, wherein:
n is 1 or 2;
the dashed lines represent optional double bonds; with the proviso that when the bond between C1 and C2 is a single bond, then R₂ and R₃ are not both hydrogen.

8. A compound of claim 1, wherein said compound is a compound of Formula VIII: 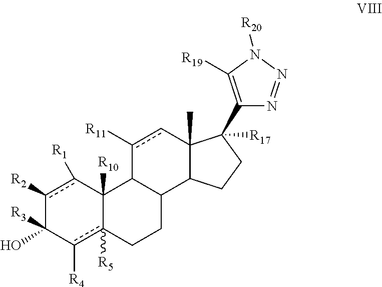
or a pharmaceutically acceptable salt, solvate, or prodrug thereof, wherein:
the dashed lines represent optional double bonds; with the proviso that when the bond between C1 and C2 is a single bond, then R₂ and R₃ are not both hydrogen.

9. A compound of claim 1, wherein said compound is a compound of Formula IX: 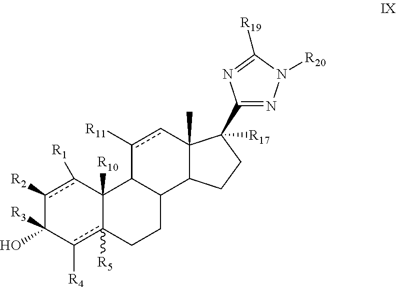
or a pharmaceutically acceptable salt, solvate, or prodrug thereof, wherein:
the dashed lines represent optional double bonds; with the proviso that when the bond between C1 and C2 is a single bond, then R₂ and R₃ are not both hydrogen.

10. A compound of claim 1, wherein said compound is a compound of Formula X: 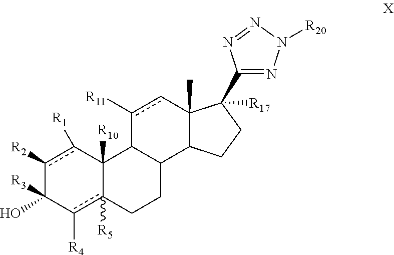
or a pharmaceutically acceptable salt, solvate, or prodrug thereof, wherein:
the dashed lines represent optional double bonds; with the proviso that when the bond between C1 and C2 is a single bond, then R₂ and R₃ are not both hydrogen.

11. A compound of claim 1, wherein said compound is a compound of Formula XI: 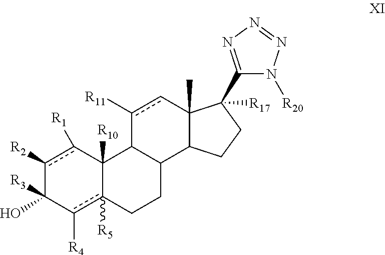
or a pharmaceutically acceptable salt, solvate, or prodrug thereof, wherein:
the dashed lines represent optional double bonds; with the proviso that when the bond between C1 and C2 is a single bond, then R₂ and R₃ are not both hydrogen.

12. A compound of claim 1, wherein said compound is a compound of Formula XII: 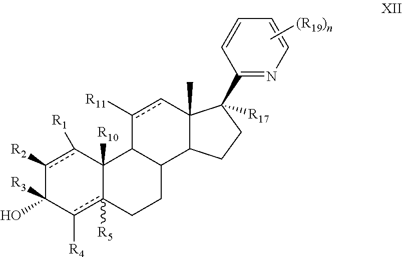
or a pharmaceutically acceptable salt, solvate, or prodrug thereof, wherein:
n is an integer from 1 to 4;
the dashed lines represent optional double bonds; with the proviso that when the bond between C1 and C2 is a single bond, then R₂ and R₃ are not both hydrogen.

13. A compound of claim 1, wherein said compound is a compound of Formula XIII: 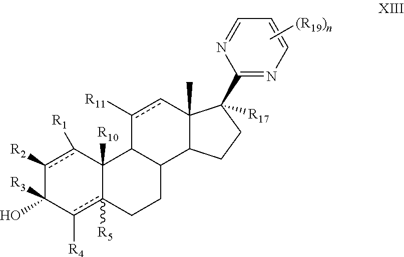
or a pharmaceutically acceptable salt, solvate, or prodrug thereof, wherein:
n is an integer from 1 to 3;
the dashed lines represent optional double bonds; with the proviso that when the bond between C1 and C2 is a single bond, then R₂ and R₃ are not both hydrogen.

14. A compound of claim 1, wherein said compound is a compound of Formula XIV: 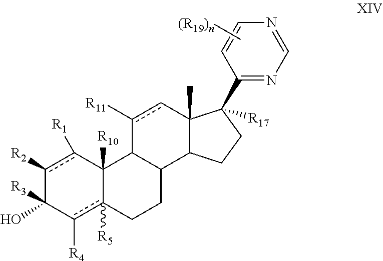
or a pharmaceutically acceptable salt, solvate, or prodrug thereof, wherein:
n is an integer from 1 to 3;
the dashed lines represent optional double bonds; with the proviso that when the bond between C1 and C2 is a single bond, then R₂ and R₃ are not both hydrogen.

15-18. (canceled)

19. A compound of claim 1, wherein R₁, R₂, R₄, R₅, R₁₇, R₂₀, R₂₂, R₂₃, R₂₄, and R₂₅ are hydrogen, R₃ is selected from the group C₁₋₄ alkyl, and C₁₋₄ haloalkyl; each R₁₉ is independently hydrogen, halogen, optionally substituted C₁₋₄ alkyl, and C₁₋₄ haloalkyl; C1 to C2, C4 to C5, and C11 to C12 are single bonds, or a pharmaceutically acceptable salts, solvates, or prodrugs thereof.

20-21. (canceled)

22. A compound of claim 1 wherein R₁, R₂, R₄, R₅, R₁₁, R₁₇, R₂₂, R₂₃, R₂₄, and R₂₅ are hydrogen; R₃ is selected from the group consisting of C₁₋₄ alkyl, C₂₋₄ alkenyl, C₂₋₄ alkynyl, and C₁₋₄ haloalkyl; R₁₀ is hydrogen or methyl; each R₁₉ is independently hydrogen, halogen, C₁₋₄ alkyl, optionally substituted with hydroxy, and halogen; HET is selected from the group consisting of 5-isoxazolyl, 3-isoxazolyl, 2-oxazolyl, 4-oxazolyl and 5-oxazolyl, all optionally substituted with 1 to 2 R₁₉ groups; C1 to C2, C4 to C5, and C11 to C12 are single bonds, a*d or a pharmaceutically acceptable salts, solvates, or prodrugs thereof.

23. A compound of claim 22 wherein R₃ is methyl; R₅ is a 5α-hydrogen atom; R₁₀ is methyl; each R₁₉ is independently hydrogen, C₁₋₄ alkyl and hydroxymethyl; or a pharmaceutically acceptable salts, solvates, or prodrugs thereof.

24. A compound of claim 1 wherein R₁, R₂, R₄, R₅, R₁₁, R₁₇, R₂₂, R₂₃, R₂₄, and R₂₅ are hydrogen; R₃ is selected from the group consisting of C₁₋₄ alkyl, C₂₋₄ alkenyl, C₂₋₄ alkynyl, and C₁₋₄ haloalkyl; R₁₀ is hydrogen or methyl; each R₁₉ is independently hydrogen, halogen, C₁₋₄ alkyl, optionally substituted with hydroxy, and halogen; HET is selected from the group consisting of 5-isoxazolyl and 3-isoxazolyl, all optionally substituted with 1 to 2 R₁₉ groups; C1 to C2, C4 to C5, and C11 to C12 are single bonds, or a pharmaceutically acceptable salts, solvates, or prodrugs thereof.

25. (canceled)

26. A compound of claim 1 wherein the compound is selected from the group of:
5-[3α-Hydroxy-3β-methyl-5α-androstan-17β-yl]-isoxazole;
3-[3α-hydroxy-3β-methyl-5α-androstan-17β-yl]-isoxazole;
ethyl 5-[3α-hydroxy-3β-methyl-5α-androstan-17β-yl]-3-isoxazolecarboxylate;
5-[3α-hydroxy-3β-methyl-5α-androstan-17β-yl]-3-(hydroxymethyl)isoxazole;
5-[3α-hydroxy-3β-methyl-5α-androstan-17β-yl]-3-isoxazolecarboxaldehyde;
(S)-3-[3α-hydroxy-3β-methyl-5α-androstan-17β-yl]-5-(1-hydroxyethyl)isoxazole;
3-[3α-hydroxy-3β-methyl-5α-androstan-17β-yl]-5-(hydroxymethyl)isoxazole;
3-[3α-hydroxy-3β-methyl-5α-androstan-17β-yl]-5-(2-hydroxy-2-propyl)isoxazole;
3-[3α-hydroxy-3β-methyl-5α-androstan-17β-yl]-5-(trimethylsilyl)isoxazole; and
2-[3α-hydroxy-3β-methyl-5α-androstan-17β-yl]-imidazo[1,2-a]pyridine; and
a pharmaceutically acceptable salts thereof.

27. A pharmaceutical composition comprising a pharmaceutically acceptable carrier or diluent and a compound of Formula I: 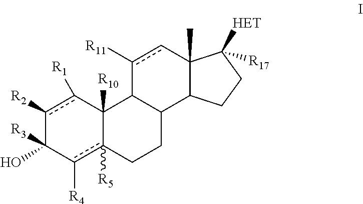
or a pharmaceutically acceptable salt, solvate, or prodrug thereof, wherein:
each R₁, R₂, R₃, R₄, and R₁₇ is independently selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁;
R₅ is a 5α or 5β-hydrogen, fluorine or absent if there is a C4-C5 double bond;
R₁₀ is hydrogen, fluorine or methyl;
R₁₁ is hydrogen, a hydroxyl, an NR₂₃R₂₄ group or a keto group;
each R₁₉ independently is hydrogen, halogen, C₁₋₈ alkyl, C₂₋₈ alkenyl, C₂₋₈ alkynyl, C₁₋₄ acyl, —C(═O)OC₁₋₄ alkyl, —C(═O)H, —Si(C₁₋₄ alkyl)₃, or C₁₋₈ haloalkyl, wherein each of said alkyl, alkenyl, alkynyl, and haloalkyl is optionally substituted with 1-5 R₂₁;
R₂₀ is selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁; or
R₂₀ is selected from the group consisting of aryl, heteroaryl, C₃₋₆ cycloalkyl, C₄₋₆ cycloalkenyl, heterocycloalkyl or heterocycloalkenyl, wherein said heterocycloalkyl is optionally fused with phenyl or a 5-6 membered heteroaryl having 1-3 heteroatoms, wherein one or more of the carbon atoms in said heterocycloalkyl or heterocycloalkenyl optionally may be oxidized to C(═O), wherein each of said aryl, heteroaryl, cycloalkyl, cycloalkenyl, heterocycloalkyl, and heterocycloalkenyl is optionally substituted with 1-5 R₂₂; or
R₁₉ and R₂₀ taken together with the atoms to which they are attached form a heteroaryl, a heterocycloalkyl or a heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), wherein each of said heteroaryl, heterocycloalkyl, and heterocycloalkenyl is optionally substituted with 1-5 R₂₂;
each R₂₁ is independently selected from the group consisting of hydroxyl, C₁₋₆ alkoxy, C₁₋₈ haloalkoxy, C₃₋₆ cycloalkoxy, NR₂₃R₂₄, aryl, heteroaryl, C₃₋₆ cycloalkyl, C₄₋₆ cycloalkenyl, heterocycloalkyl or heterocycloalkenyl, wherein each of said aryl, heteroaryl, cycloalkyl and cycloalkenyl are optionally substituted with 1-5 R₂₂; and wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), and wherein each of said heterocycloalkyl and said heterocycloalkenyl is optionally substituted with 1-5 R₂₂;
each R₂₂ is independently selected from the group consisting of nitro, nitrile, hydroxyl, halogen, C₁₋₆ acyl, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₆ cycloalkoxy, aryl, heteroaryl, —NR₂₃R₂₄, —C(═O)OR₂₃, —C(═O)NHR₂₃, —NHC(═O)R₂₅, —NHS(═O)₂R₂₅, —S(═O)₀₋₂R₂₅, —S(═O)₂NHR₂₃, C₃₋₆ cycloalkyl, C₄₋₆ cycloalkenyl, heterocycloalkyl or heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O);
each of R₂₃ and R₂₄ is independently hydrogen, C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted with 1-5 R₂₁;
R₂₅ is C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted;
HET is a heteroaryl group selected from 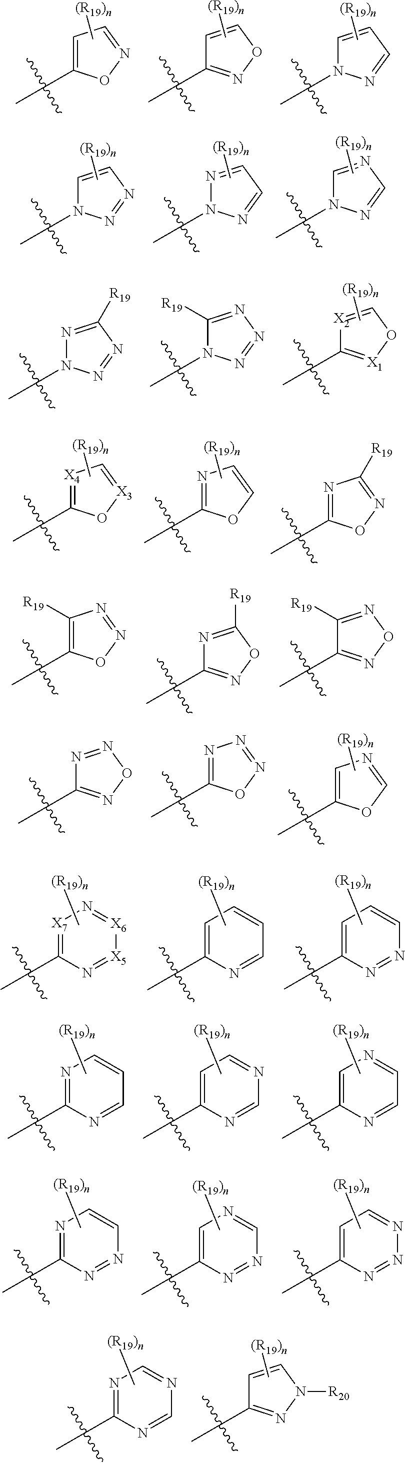 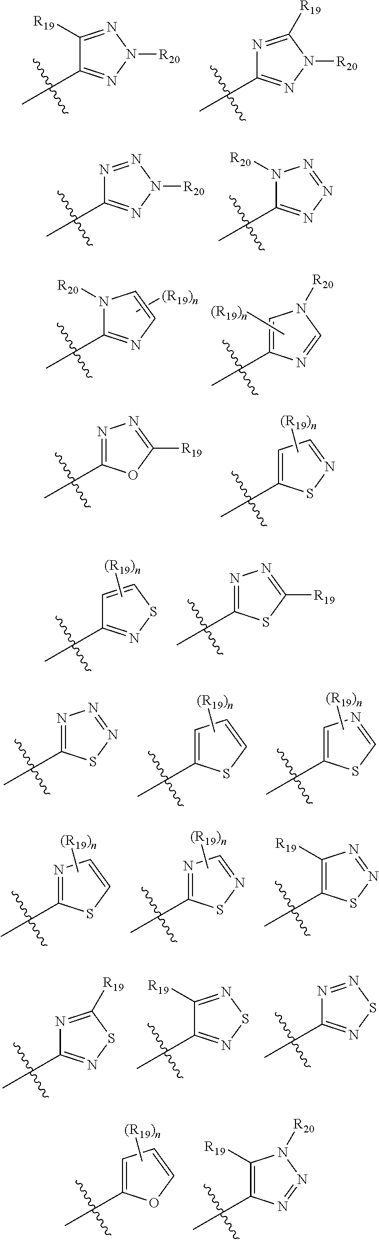
when X₁ is N, X₂ is CR₁₉ or N, when X₁ is CR₁₉, X₂ is N;
X₃ and X₄ are independently CR₁₉ and N;
when X₅ is N, X₆ and X₇ are independently CR₁₉; or
when X₆ is N, X₅ and X₇ are independently CR₁₉; or
when X₇ is N, X₅ and X₆ are independently CR₁₉;
n is an integer from 1 to 4;
the dashed lines represent optional double bonds; with the proviso that when the bond between C1 and C2 is a single bond, then R₂ and R₃ are not both hydrogen.

28. A method for treating a patient having a CNS disorder amenable to modulation of the GABA_{A} receptor-chloride channel ionophore comprising administering to a patient in need of such treatment a pharmaceutical composition comprising a pharmaceutically acceptable carrier or diluent and a compound of Formula I: 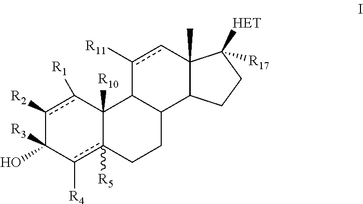
or a pharmaceutically acceptable salt, solvate, or prodrug thereof, wherein:
each R₁, R₂, R₃, R₄, and R₁₇ is independently selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁;
R₅ is a 5α or 5β-hydrogen, fluorine or absent if there is a C4-C5 double bond;
R₁₀ is hydrogen, fluorine or methyl;
R₁₁ is hydrogen, a hydroxyl, an NR₂₃R₂₄ group or a keto group;
each R₁₉ independently is hydrogen, halogen, C₁₋₈ alkyl, C₂₋₈ alkenyl, C₂₋₈ alkynyl, C₁₋₄ acyl, —C(═O)OC₁₋₄ alkyl, —C(═O)H, —Si(C₁₋₄ alkyl)₃, or C₁₋₈ haloalkyl, wherein each of said alkyl, alkenyl, alkynyl, and haloalkyl is optionally substituted with 1-5 R₂₁;
R₂₀ is selected from the group consisting of hydrogen, C₁₋₈ alkyl, C₂₋₈ alkenyl, C₂₋₈ alkynyl, and C₁₋₈ haloalkyl, wherein each of said alkyl, alkenyl, alkynyl, and haloalkyl is optionally substituted with 1-5 R₂₁; or
R₂₀ is selected from the group consisting of aryl, heteroaryl, C₃₋₆ cycloalkyl, C₄₋₆ cycloalkenyl, heterocycloalkyl or heterocycloalkenyl, wherein said heterocycloalkyl is optionally fused with phenyl or a 5-6 membered heteroaryl having 1-3 heteroatoms, wherein one or more of the carbon atoms in said heterocycloalkyl or heterocycloalkenyl optionally may be oxidized to C(═O), wherein each of said aryl, heteroaryl, cycloalkyl, cycloalkenyl, heterocycloalkyl, and heterocycloalkenyl is optionally substituted with 1-5 R₂₂; or
R₁₉ and R₂₀ taken together with the atoms to which they are attached form a heteroaryl, a heterocycloalkyl or a heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), wherein each of said heteroaryl, heterocycloalkyl, and heterocycloalkenyl is optionally substituted with 1-5 R₂₂;
each R₂₁ is independently selected from the group consisting of hydroxyl, C₁₋₆ alkoxy, C₁₋₈ haloalkoxy, C₃₋₆ cycloalkoxy, NR₂₃R₂₄, aryl, heteroaryl, C₃₋₆ cycloalkyl, C₄₋₆ cycloalkenyl, heterocycloalkyl or heterocycloalkenyl, wherein each of said aryl, heteroaryl, cycloalkyl and cycloalkenyl are optionally substituted with 1-5 R₂₂; and wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O), and wherein each of said heterocycloalkyl and said heterocycloalkenyl is optionally substituted with 1-5 R₂₂;
each R₂₂ is independently selected from the group consisting of nitro, nitrile, hydroxyl, halogen, C₁₋₆ acyl, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₆ cycloalkoxy, aryl, heteroaryl, —NR₂₃R₂₄, —C(═O)OR₂₃, —C(═O)NHR₂₃, —NHC(═O)R₂₅, —NHS(═O)₂R₂₅, —S(═O)₀₋₂R₂₅, —S(═O)₂NHR₂₃, C₃₋₆ cycloalkyl, C₄₋₆ cycloalkenyl, heterocycloalkyl or heterocycloalkenyl, wherein said heterocycloalkyl is optionally fused with a phenyl or a 5-6 membered heteroaryl having 1-3 heteroatoms, wherein one or more of the carbon atoms in said heterocycloalkyl or heterocycloalkenyl optionally may be oxidized to C(═O);
each of R₂₃ and R₂₄ is independently hydrogen, C₁₋₆ alkyl, C₂₋₆ alkenyl, C₂₋₆ alkynyl, C₁₋₆ haloalkyl, C₃₋₆ cycloalkyl or C₄₋₆ cycloalkenyl; wherein each of said alkyl, alkenyl, alkynyl, haloalkyl, cycloalkyl and cycloalkenyl is optionally substituted with 1-5 R₂₁;
R₂₅ is C₁₋₆ alkyl, C₂₋₆ alkenyl, C₂₋₆ alkynyl, C₁₋₆ haloalkyl, C₃₋₆ cycloalkyl or C₄₋₆ cycloalkenyl;
wherein each of said alkyl, alkenyl, alkynyl, haloalkyl, cycloalkyl and cycloalkenyl is optionally substituted;
HET is a heteroaryl group selected from 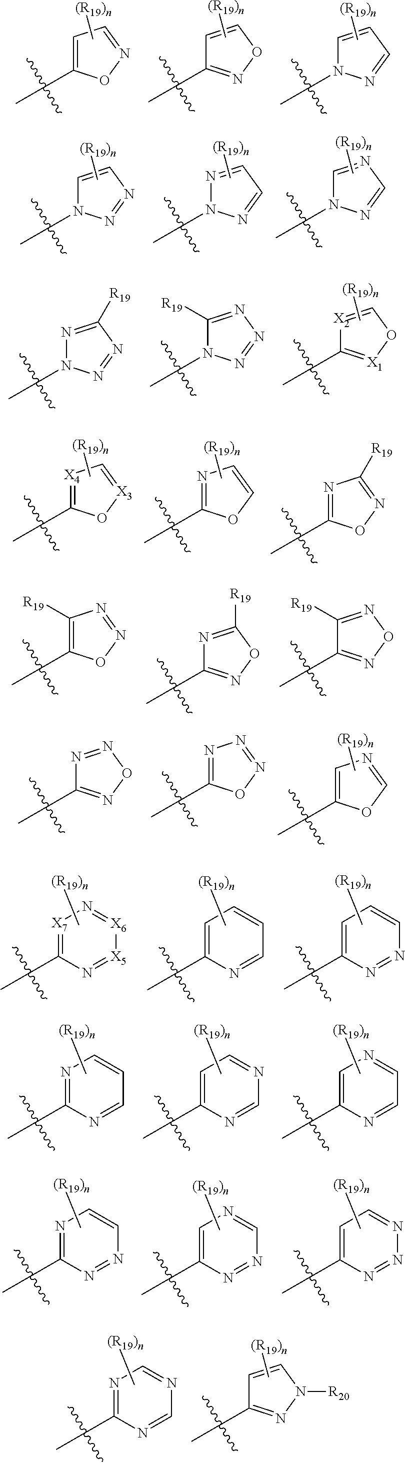 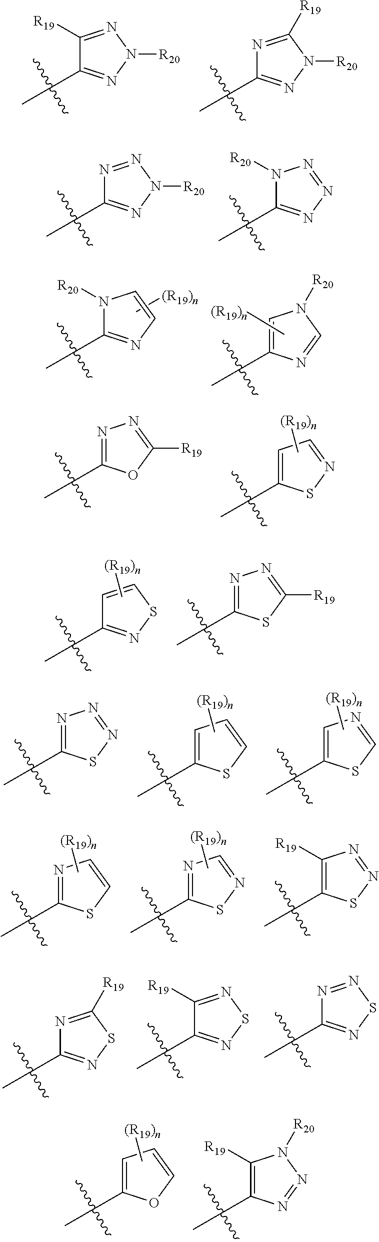
when X₁ is N, X₂ is CR₁₉ or N, when X₁ is CR₁₉, X₂ is N;
X₃ and X₄ are independently CR₁₉ and N;
when X₅ is N, X₆ and X₇ are independently CR₁₉; or
when X₆ is N, X₅ and X₇ are independently CR₁₉; or
when X₇ is N, X₅ and X₆ are independently CR₁₉;
n is an integer from 1 to 4;
the dashed lines represent optional double bonds.

29. The method of claim 28, wherein said CNS disorder is an anxiety disorder, a seizure disorder, an affective disorder, a sleep disorder or an autism spectrum disorder.

30-35. (canceled)

36. The method of claim 28, wherein the CNS disorder is convulsions, depression, insomnia, chronic pain, acute pain or multiple sclerosis.

37-46. (canceled)

47. A compound according to claim 1, wherein HET is a heteroaryl group selected from [chemical structure]
